(19) United States
(12) Patent Application Publication
(10) Pub. No.: US 2014/0045805 A1
(43) Pub. Date: Feb. 13, 2014

(54) Fragrance Compositions and Other Compositions Which Contain Naturally Occurring Substances Found in Corals

(71) Applicant: Human Pheromone Sciences, Inc., (US)

(72) Inventors: Chloe Jennings-White, West Bountiful, UT (US); Louis Monti-Bloch, Mountain View, CA (US)

(73) Assignee: Human Pheromone Sciences, Inc., San Jose, CA (US)

(21) Appl. No.: 13/851,057

(22) Filed: Mar. 26, 2013

Related U.S. Application Data
(63) Continuation of application No. 12/211,448, filed on Sep. 16, 2008, now abandoned.
(60) Provisional application No. 60/972,998, filed on Sep. 17, 2007.

(51) Int. Cl. A61K 31/57

(52) U.S. Cl. CPC A61K 31/57
USPC 514/177; 514/182

(57) ABSTRACT

This invention is generally related to the fields of fragrance compositions, personal care products, and home consumer products. This invention also relates to 20-pregnenes, in particular those found naturally occurring in corals and which affect mood in humans, to the incorporation of these 20-pregnene compounds into various compositions, and to methods of affecting the mood of individuals using such compounds.

### CROSS REFERENCE TO RELATED APPLICATIONS

This application claims the benefit of U.S. Provisional Application No. 60/972,998, filed Sep. 17, 2007, the contents of which are hereby incorporated by reference in their entirety.

### FIELD OF THE INVENTION

This invention is generally related to the fields of fragrance compositions, personal care products, and home consumer products. This invention also relates to 20-pregnenes, in particular those found naturally occurring in corals and which affect mood in humans, to the incorporation of these 20-pregnene compounds into various compositions, and to methods of affecting the mood of individuals using such compounds.

### BACKGROUND OF THE INVENTION

Marine pregnenes are substances from the pregnene family which have at least one double bond, and which occur naturally in organisms that dwell in the sea. Pregna-5,20-dien-3β-ol, also known as muricin aglycone, has been described as a constituent of the Alyconacean coral Gersemia rubiformis, commonly referred to as sea raspberry, inhabiting the cold waters off the coast of Newfoundland and Labrador (Kingston et al., J. C. S. Perkin I, 1979, 2064). Muricin aglycone also occurs in the coral Carijoa riisei (Telestacea) off the coast of the Brazilian island of Guaibinha (Maia et al., Bol. Soc. Chil. Quim., 1998, 43(1), 39). Muricin aglycone also occurs in the South African soft coral, Pieterfaurea unilobata (Beukes et al., J. Nat. Prod., 1997, 60, 573). In addition, muricin itself, wherein the steroid moiety is conjugated with a sugar, occurs in the gorgonian coral, Muricea fruticosa, off the coast of California (Bandurraga and Fenical, Tetrahedron, 1985, 41(6), 1057). Pregna-1,4,20-trien-3-one, pregna-1,20-dien-3-one, and pregn-20-en-3α-yl acetate have been found to occur in an unidentified soft coral off the coast of Canton Island (Higgs and Faulkner, Steroids, 1977, 30(3), 379). Pregna-1,4,20-trien-3-one also occurs in the Alyconacean coral Gersemia rubiformis (Kingston et al., J. C. S. Perkin I, 1979, 2064), and is the major steroidal component of the coral Carijoa riisei (Telestacea) (Maia et al., Bol. Soc. Chil. Quim., 1998, 43(1), 39).

Materials derived from marine organisms, such as ambergris, have been used in perfumery as odorants and/or fixatives. However, they are not known to produce a particular psychological effect. Muricin aglycone has been subject to testing for biological activity. For example, muricin aglycone was evaluated as an inhibitor of human testicular 17α Hydroxylase/C₁₇₋₂₀-Lyase (Li et al., J. Med. Chem. 1996, 39, 4335; Brodie et al. U.S. Pat. No. 5,994,334, 1999). Compounds with a hydrophobic D-ring, including muricin aglycone, showed little activity in this assay. Likewise, muricin aglycone has been tested for inhibition of rabbit and rat cytochrome P-450 (Halperper sect et al., Mol. Pharmacol., 1989, 35(1), 148; Klekotka et al., J. Pharmacol. Exp. Ther., 1995, 274(3), 1450). Muricin aglycone has been found to elicit cross adaptation with androsta-4,16-dien-3-one in the local electrical response of the human vomeronasal epithelium (Monti-Bloch et al., Psychoneuroendocrinology 1994, 19, 673). Androsta-4,16-dien-3-one has been found to produce a reduction of negative affect when administered nasally to humans (Grosser et al., Psychoneuroendocrinology, 2000, 25, 289).

As has been the case for ambergris from the sperm whale, the harvesting of the naturally occurring 20-pregnenes from coral is neither efficient, nor cost effective, nor ecologically sound. Consequently, as is the case for ambergris, a synthetic approach is desirable. The synthesis of several of these 20-pregnenes has been described in the literature. Pregnenolone hydrazone was treated with iodine and triethylamine to give the vinyl iodide, which was then treated with sodium and ethanol to give pregna-5,20-dien-3β-ol (Krubiner et al., J. Org. Chem., 1969, 34, 11, 3502). Pregna-5,20-dien-3β-ol has also been synthesized from pregnenolone acetate by conversion to the corresponding alcohol, from which the mesylate was prepared followed by treatment with potassium t-butoxide (Dawe and Wright, Can. J. Chem., 1987, 65, 666). The first described synthesis of pregna-5,20-dien-3β-ol started with 3-acetoxybisnorcholenic acid. This was subjected to Curtius rearrangement, N-methylation, and Hofmann degradation (Julian et al., J. Amer. Chem. Soc., 1948, 70, 887). Pregna-5,20-dien-3β-ol was synthesized from pregnenolone by converting to the ethylene thioketal, and then treating with Raney nickel (Kingston et al., J. C. S. Perkin I, 1979, 2064). These same authors used similar methodology for synthesizing pregna-1,4,20-trien-3-one, also in the Sea Raspberry, from progesterone. Pregna-5,20-dien-3β-ol may also be synthesized by the treatment of pregnenolone tosylhydrazone with methyl lithium (Bose and Steinberg, Synthesis, 1970, 595). 5α-pregna-1,20-dien-3-one, as well as 5α-pregn-20-en-3β-ol, have also been synthesized via the same tosylhydrazone method (Schow and McMorris, Steroids, 1977, 30, 389).

U.S. Pat. No. 5,272,134 relates to fragrance compositions and other compositions which contain human pheromones.

U.S. Pat. No. 5,278,141 relates to fragrance compositions containing human pheromones.

U.S. Pat. No. 5,563,131 relates to pregnane steroids as neurochemical initiators of change in human hypothalamic function.

U.S. Pat. No. 5,994,333 relates to pregnane and cholane steroids as neurochemical initiators of change in human hypothalamic function.

U.S. Pat. No. 5,994,334 relates to androgen synthesis inhibitors.

U.S. Pat. No. 6,242,619 relates to 19-norpregnane steroids as neurochemical initiators of change in human hypothalamic function.

U.S. Pat. No. 6,277,838 relates to methods for allosteric modulation of the GABA receptor by members of the androstane and pregnane series.

U.S. Pat. No. 6,331,534 relates to steroids as neurochemical stimulators of the vomeronasal organ to alleviate pain.

What is needed are methods for affecting the mood of individuals, and compositions for use in such methods. What is further needed is an improved method for synthesizing 20-pregnene compounds.

### SUMMARY OF THE INVENTION

Provided herein are 20-pregnene compounds that have an affect on human mood, as well as compositions containing and methods of using such compounds.

In one aspect of the invention are provided methods of affecting the mood of an individual, the method comprising nasal administration of an effective amount of at least one 20-Pregnene compound having the formula:

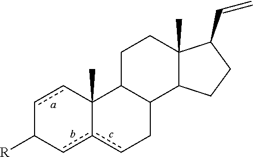

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond; and
wherein the effect on the individual's mood comprises at least one of the following: an increase in the individual's social warmth and friendliness, an increase in the individual's arousal/excitement, or an increase in the individual's sensuality.

In some embodiments of the methods, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In certain embodiments, the at least one compound is pregna-1,4,20-trien-3-one.

In some embodiments, the effect on the individual's mood includes an increase in the individual's social warmth and friendliness. In certain embodiments, the effect on the individual's mood includes an increase in the individual's perception of loving level, and the individual is female. In some embodiments, the effect on the individual's mood includes an increase in the individual's perception of warmth, and wherein the individual is female.

In some embodiments, the effect on the individual's mood includes an increase in the individual's arousal/excitement. In particular embodiments, the effect on the individual's mood includes an increase in the individual's perception of excited level, and wherein the individual is female. In some embodiments, the effect on the individual's mood includes an increase in the individual's perception of excited level, and the individual is male. In some embodiments, the effect on the individual's mood includes an increase in the individual's perception of energetic level, and the individual is male.

In some embodiments, the effect on the individual's mood includes an increase in the individual's sensuality.

In some embodiments, the effect on the individual's mood includes an increase in the individual's personal well-being, and the individual is female.

Some embodiments of the methods include delivering about 50 picograms to about 100 nanograms of the at least one compound to the individual. In some embodiments of the methods include delivering about 250 picograms to about 100 nanograms of the at least one compound to the individual. In some embodiments, the methods include delivering the at least one compound to the individual over a time period of about 1 minute to about 18 hours. In some embodiments, the compound is delivered over a time period of less than 1 minute. In some embodiments, the compound is delivered over a time period of about 30 seconds to about 8 hours. In some embodiments, the methods include delivering the at least one compound to the individual over a time period of about 6 minutes to about 18 hours. In some embodiments, the methods include delivering the at least one compound to the individual over a time period of about 20 minutes to about 18 hours.

In some embodiments, the at least one compound is delivered to the individual in a composition and the total concentration of the at least one 20-Pregnene compound(s) in the composition is about 100 ng/ml to about 1 mg/ml. In some embodiments, the total concentration of the compound in the composition is from about 1 ng/ml to about 50 mg/ml. In some embodiments, the total concentration of the at least one 20-Pregnene compound(s) in the composition is about 1 μg/ml to about 100 μg/ml. In some embodiments the total concentration is about 100 ng/ml to about 1 mg/ml.

In some embodiments, the at least one compound is delivered in a 20-pregnene containing composition that is non-therapeutic.

In some embodiments, the at least one compound is delivered in a fragrance composition, wherein the fragrance composition includes an odorant and the at least one compound. In some embodiments, the fragrance composition is formulated for external application to the skin. In some embodiments, the fragrance composition is a perfume, a mist, a perfumed wax, an eau de toilette, or cologne.

In some embodiments, the at least one compound is delivered in a personal care product selected from the group consisting of: a cosmetic, a hair care product, a bath or shower product, a skin care product, an antiperspirant, a deodorant, a massage oil, a nail product, and a personal lubricant. In some embodiments, the personal care product further includes at least one odorant. In some embodiments, the personal care product is a cosmetic formulated for application to the skin of the face, the body, the lips, the eyelids, the eyelashes, or the nails. In some embodiments, the personal care product is a hair care product selected from the group consisting of: a shampoo, a conditioner, a hair gel, a hair treatment product, hair dye, pomade, and a hairspray. In some embodiments, the personal care product is a bath or shower product selected from the group consisting of: a soap, a non-soap cleanser, a gel, a foam, and a lotion. In some embodiments, the personal care product is a skin care product selected from the group consisting of: a moisturizer, a shaving cream, a shaving lotion, a shaving gel, an aftershave lotion, an aftershave cream, an aftershave balm, a topical analgesic, a skin bronzer, a skin lightening agent, and a sunscreen. In some embodiments, the personal care product is an OTC (over-the-counter) or non-prescription dermatological product. In some embodiments, the personal care product is a masculine lubricant or a feminine lubricant. In some embodiments, the personal care product is a personal lubricant, and the lubricant has been pre-applied to a condom. In some embodiment, the at least one compound is delivered in a personal care product that is an unscented product sprayed or applied to the skin.

In some embodiments, the at least one compound is delivered in a composition including a fibrous material and the at least one compound. In some embodiments, the composition further includes an odorant. In some embodiments, the composition is selected from a fabric or a paper product. In some embodiments, the composition is a tissue. In some embodiments, the composition is selected from a bandage, a surgical tape, and elastic fabric support. In some embodiments the composition is a wipe.

In some embodiments, the at least one compound is delivered in a composition including a polymeric component and the at least one compound. In some embodiments, the polymeric material includes a polymer occurring in nature. In some embodiments, the polymeric material includes a synthetic polymer.

In some embodiments, the at least one compound is delivered in a fabric that has been treated with a laundry product including the at least one compound. In some embodiments, the laundry product is selected from the group consisting of: a liquid or powdered laundry detergent, liquid or powdered laundry soap, a bleach, a stain remover, and a fabric softener. In some embodiments, the laundry product further includes an odorant.

In some embodiments, the at least one compound is delivered in an environmental product selected from a room freshener, a car freshener, or a candle. In some embodiments, the environmental product further includes an odorant.

In some embodiments, the at least one compound is delivered in a cleaning product. In some embodiments, the cleaning product is selected from a dishwashing soap or detergent, a disinfectant, a sterilizer, a floor cleaner, an appliance cleaner, a glass cleaner, a tile cleaner, a laminate cleaner, and a stone cleaner. In some embodiments, the cleaning product further includes an odorant.

In some embodiments, the at least one compound is delivered by a nasal inhaler.

In another aspect are provided 20-Pregene-containing compositions comprising at least one 20-Pregnene compound having the formula:

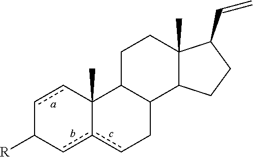

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond; and a composition selected from the group consisting of fragrance compositions comprising an odorant, personal care products, compositions comprising a fibrous material or polymeric component, laundry products, cleaning products, and environmental products.

In some embodiments, the 20-Pregnene-containing composition is formulated to release the at least one compound for nasal delivery to an individual in an amount effective to affect the mood of the individual; and the effect on the individual's mood comprises at least one of the following: an increase in the individual's social warmth and friendliness, an increase in the individual's arousal/excitement, or an increase in the individual's sensuality. In some embodiments of the 20-Pregenene-containing compositions the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one. In some embodiments, the composition is a fragrance composition. In some embodiments, the 20-pregnene compositions are non-therapeutic.

Thus in some embodiments of the present invention are provided fragrance compositions including an odorant and at least one 20-Pregnene compound having the formula:

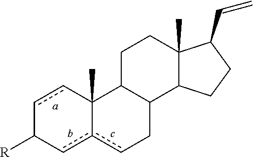

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond.

In some embodiments, the fragrance composition is formulated to release the at least one compound for nasal delivery to an individual in an amount effective to affect the mood of the individual; wherein the effect on the individual's mood comprises at least one of the following: an increase in the individual's social warmth and friendliness, an increase in the individual's arousal/excitement, or an increase in the individual's sensuality. In some embodiments, the fragrance composition is non-therapeutic.

In some embodiments of the fragrance compositions, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one; In some embodiments, the at least one compound is pregna-5,20-dien-3β-ol. In some embodiments, the at least one compound is pregna-1,4,20-trien-3-one.

In some embodiments of the fragrance compositions, the effect on the individual's mood includes an increase in the individual's social warmth and friendliness. In some embodiments, the effect on the individual's mood comprises an increase in the individual's perception of loving level, and wherein the individual is female. In some embodiments, the effect on the individual's mood comprises an increase in the individual's perception of warmth level, and wherein the individual is female.

In some embodiments, the effect on the individual's mood includes an increase in the individual's arousal/excitement. In some embodiments, the effect on the individual's mood includes an increase in the individual's perception of excited level, and the individual is female. In some embodiments, the effect on the individual's mood includes an increase in the individual's perception of excited level and the individual is male. In some embodiments, the effect on the individual's mood comprises an increase in the individual's perception of energetic level and the individual is male.

In some embodiments of the fragrance compositions, the effect on the individual's mood includes an increase in the individual's sensuality.

In some embodiments of the fragrance compositions, the effect on the individual's mood includes an increase in the individual's personal well-being and the individual is female.

In some embodiments of the fragrance compositions, the fragrance composition is formulated for external application to the skin. In some embodiments of the fragrance compositions, the fragrance composition is a perfume, a mist, a perfumed wax, eau de toilette, or cologne.

In some embodiments of the fragrance compositions, the compound is present in the composition at a concentration of about 1 ng/ml to about 50 mg/ml. In some embodiments, the compound is present in the composition at a concentration of about 100 ng/ml to about 1 mg/ml. In some embodiments, the compound is present in the composition at a concentration of about 10 μg/ml.

In some embodiments of the fragrance compositions, the total amount of all odorants comprises about 12% by weight of the composition.

In embodiment of the present invention are provided personal care products including a personal care product and at least one 20-Pregnene compound having the formula:

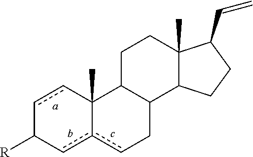

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond.

In some embodiments of the personal care products, the personal care product is formulated to release the at least one compound for nasal delivery to an individual in an amount effective to affect the mood of the individual and the effect on the individual's mood comprises at least one of the following: an increase in the individual's social warmth and friendliness, an increase in the individual's arousal/excitement, or an increase in the individual's sensuality. In some embodiments, the personal care product is non-therapeutic.

In some embodiments of the personal care products, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one.

In some embodiments of the personal care products, the effect on the individual's mood includes an increase in the individual's social warmth and friendliness. In some embodiments of the personal care products, the effect on the individual's mood includes an increase in the individual's perception of loving level and the individual is female. In some embodiments of the personal care products, the effect on the individual's mood includes an increase in the individual's perception of warmth level and the individual is female.

In some embodiments of the personal care products, the effect on the individual's mood comprises an increase in the individual's arousal/excitement. In some embodiments, the effect on the individual's mood comprises an increase in the individual's perception of excited level and the individual is female. In some embodiments, the effect on the individual's mood includes an increase in the individual's perception of excited level and the individual is male. In some embodiments, the effect on the individual's mood comprises an increase in the individual's perception of energetic level and the individual is male.

In some embodiments of the personal care products, the effect on the individual's mood includes an increase in the individual's sensuality.

In some embodiments of the personal care products, the effect on the individual's mood includes an increase in the individual's personal well-being and the individual is female.

In some embodiments of the personal care products, the personal care product is selected from the group consisting of: a cosmetic, a hair care product, a bath or shower product, a skin care product, an antiperspirant, a deodorant, a massage oil, a nail product, and a personal lubricant.

In some embodiments, the product further includes an odorant.

In some embodiments of the personal care products, the personal care product is a cosmetic formulated for application to the skin the face, the body, the lips, the eyelids, the eyelashes, or the nails.

In some embodiments, the personal care product is a hair care product selected from the group consisting of: a shampoo, a conditioner, a hair gel, a hair treatment product, hair dye, a pomade, and a hairspray.

In some embodiments, the personal care product is a bath or shower product selected from the group consisting of: a soap, non-soap cleanser, a gel, a foam, and a lotion.

In some embodiments, the personal care product is a skin care product selected from the group consisting of: a moisturizer, a shaving cream, a shaving lotion, a shaving gel, an aftershave lotion, an aftershave cream, an aftershave balm, a topical analgesic, a skin bronzer, a skin lightening agent, and a sunscreen.

In some embodiments, the personal care product is an OTC or non-prescription dermatological product.

In some embodiments, the personal care product is an unscented product sprayed or applied to the skin.

In some embodiments, the personal care product is a masculine lubricant or a feminine lubricant. In some embodiments, the personal care product is a personal lubricant, wherein the lubricant has been pre-applied to a condom.

In another embodiment are presented compositions including a fibrous material or polymeric component and at least one 20-Pregnene compound having the formula:

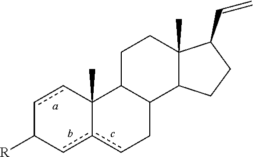

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond.

In some embodiments, the composition is formulated to release the at least one compound for nasal delivery to an individual in an amount effective to affect the mood of the individual; and the effect on the individual's mood comprises at least one of the following: an increase in the individual's social warmth and friendliness, an increase in the individual's arousal/excitement, or an increase in the individual's sensuality. In some embodiments, the composition is non-therapeutic.

In some embodiments of the compositions, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one.

In some embodiments of the compositions, the effect on the individual's mood includes an increase in the individual's social warmth and friendliness. In some embodiments, the effect on the individual's mood includes an increase in the individual's perception of loving level and the individual is female. In some embodiments, the effect on the individual's mood includes an increase in the individual's perception of warmth level, and the individual is female.

In some embodiments of the compositions, the effect on the individual's mood includes an increase in the individual's arousal/excitement.

In some embodiments of the compositions, the effect on the individual's mood includes an increase in the individual's perception of excited level, and the individual is female. In some embodiments, the effect on the individual's mood includes an increase in the individual's perception of excited level, and the individual is male. In some embodiments, the effect on the individual's mood includes an increase in the individual's perception of energetic level, and the individual is male.

In some embodiments of the compositions, the effect on the individual's mood comprises an increase in the individual's sensuality.

In some embodiments of the compositions, the effect on the individual's mood comprises an increase in the individual's personal well-being, and the individual is female.

In some embodiments of the compositions, the composition further includes an odorant.

In some embodiments of the compositions, the composition comprises a fibrous material. In some embodiments, the composition is selected from a fabric or a paper product. In some embodiments, the composition is a tissue or wipe. In some embodiments, the composition is a tissue. In some embodiments, the composition is a wipe.

In some embodiments of the compositions, the composition is selected from a bandage, a surgical tape and an elastic fabric support.

In some embodiments of the compositions, the composition includes a polymeric material. In some embodiments, the polymeric material includes a polymer occurring in nature. In some embodiments of the compositions, the polymeric material includes a synthetic polymer.

In some embodiments of the compositions, the composition is selected from an adhesive wound covering and a surgical tape.

In yet another embodiment of the present invention are provided laundry products including a laundry product and at least one 20-Pregnene compound having the formula:

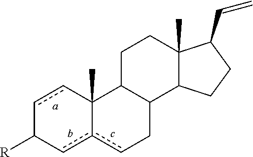

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond; and

In some embodiments of the laundry products, the laundry product is formulated to impregnate a fabric with the at least one compound for nasal delivery to an individual in an amount effective to affect the mood of the individual and the effect on the individual's mood comprises at least one of the following: an increase in the individual's social warmth and friendliness, an increase in the individual's arousal/excitement, or an increase in the individual's sensuality.

In some embodiments of the laundry products, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one.

In some embodiments of the laundry products, the effect on the individual's mood includes an increase in the individual's social warmth and friendliness. In some embodiments, the effect on the individual's mood includes an increase in the individual's perception of loving level, and the individual is female. In some embodiments of the laundry products, the effect on the individual's mood includes an increase in the individual's perception of warmth level, and the individual is female.

In some embodiments of the laundry products, the effect on the individual's mood includes an increase in the individual's arousal/excitement. In some embodiments, the effect on the individual's mood includes an increase in the individual's perception of excited level, and the individual is female. In some embodiments, the effect on the individual's mood includes an increase in the individual's perception of excited level, and the individual is male. In some embodiments, the effect on the individual's mood includes an increase in the individual's perception of energetic level, and the individual is male.

In some embodiments of the laundry products, the effect on the individual's mood includes an increase in the individual's sensuality.

In some embodiments of the laundry products, the effect on the individual's mood includes an increase in the individual's personal well-being, and the individual is female.

In some embodiments of the laundry products, the laundry product further includes an odorant.

In some embodiments of the laundry products, the laundry product is selected from the group consisting of: a liquid or powdered laundry detergent, a liquid or powdered laundry soap, a bleach, a stain remover, and a fabric softener.

In another embodiment of the invention are provided environmental products including an environmental product and at least one 20-Pregnene compound having the formula:

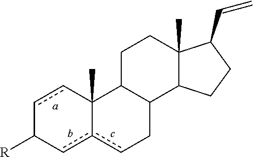

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond; and

In some embodiments of the environmental product, the environmental product is formulated to release the at least one compound for nasal delivery to an individual in an amount effective to affect the mood of the individual and the effect on the individual's mood comprises at least one of the following: an increase in the individual's social warmth and friendliness, an increase in the individual's arousal/excitement, or an increase in the individual's sensuality.

In some embodiments of the environmental products, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one.

In some embodiments of the environmental products, the effect on the individual's mood includes an increase in the individual's social warmth and friendliness. In some embodiments, the effect on the individual's mood includes an increase in the individual's perception of loving level, and the individual is female. In some embodiments, the effect on the individual's mood includes an increase in the individual's perception of warmth level, and the individual is female.

In some embodiments of the environmental products, the effect on the individual's mood includes an increase in the individual's arousal/excitement. In some embodiments, the effect on the individual's mood includes an increase in the individual's perception of excited level, and the individual is female. In some embodiments, the effect on the individual's mood includes an increase in the individual's perception of excited level, and the individual is male. In some embodiments of the environmental products, the effect on the individual's mood includes an increase in the individual's perception of energetic level, and the individual is male.

In some embodiments of the environmental products, the effect on the individual's mood includes an increase in the individual's sensuality.

In some embodiments of the environmental products, the effect on the individual's mood includes an increase in the individual's personal well-being, and the individual is female.

In some embodiments of the environmental products, the environmental product further includes an odorant.

In some embodiments of the environmental products, the environmental product is selected from a room freshener, a car freshener, or a candle.

In a further embodiment of the invention are provided cleaning products include a cleaning product and at least one 20-Pregnene compound having the formula:

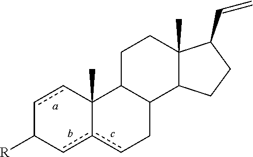

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond.

In some embodiments of the cleaning product, the cleaning product is formulated to release the at least one compound for nasal delivery to an individual in an amount effective to affect the mood of the individual and wherein the effect on the individual's mood comprises at least one of the following: an increase in the individual's social warmth and friendliness, an increase in the individual's arousal/excitement, or an increase in the individual's sensuality.

In some embodiments of the cleaning products, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one.

In some embodiments of the cleaning products, the effect on the individual's mood includes an increase in the individual's social warmth and friendliness. In some embodiments, the effect on the individual's mood includes an increase in the individual's perception of loving level, and the individual is female. In some embodiments, the effect on the individual's mood includes an increase in the individual's perception of warmth level, and the individual is female.

In some embodiments of the cleaning products, the effect on the individual's mood includes an increase in the individual's arousal/excitement. In some embodiments, the effect on the individual's mood includes an increase in the individual's perception of excited level, and the individual is female. In some embodiments, the effect on the individual's mood includes an increase in the individual's perception of excited level, and the individual is male. In some embodiments of the cleaning products, the effect on the individual's mood includes an increase in the individual's perception of energetic level, and the individual is male.

In some embodiments of the cleaning products, the effect on the individual's mood includes an increase in the individual's sensuality.

In some embodiments of the cleaning products, the effect on the individual's mood includes an increase in the individual's personal well-being, and the individual is female.

In some embodiments of the cleaning products, the cleaning product further includes an odorant.

In some embodiments of the cleaning products, the cleaning product is selected from a dishwashing soap or detergent, a disinfectant, a sterilizer, a floor cleaner, an appliance cleaner, a glass cleaner, a tile cleaner, a laminate cleaner, and a stone cleaner.

In a further aspect of the invention are provided nasal delivery devices containing at least one 20-Pregnene compound having the formula:

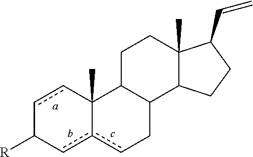

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond; wherein the nasal delivery device is designed to release directly into the nasal cavity of an individual; and wherein the at least one 20-pregenene compound is present in a non-therapeutic formulation.

In some embodiments of the nasal delivery devices, the at least one compound for nasal delivery to the individual in an amount effective to affect the mood of the individual; and the effect on the individual's mood comprises at least one of the following: an increase in the individual's social warmth and friendliness, an increase in the individual's arousal/excitement, or an increase in the individual's sensuality.

In some embodiments of the nasal delivery devices, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one.

In some embodiments of the nasal delivery devices, the effect on the individual's mood includes an increase in the individual's social warmth and friendliness. In some embodiments, the effect on the individual's mood includes an increase in the individual's perception of loving level, and the individual is female. In some embodiments, the effect on the individual's mood includes an increase in the individual's perception of warmth level, and the individual is female.

In some embodiments of the nasal delivery devices, the effect on the individual's mood includes an increase in the individual's arousal/excitement. In some embodiments, the effect on the individual's mood includes an increase in the individual's perception of excited level, and the individual is female. In some embodiments, the effect on the individual's mood includes an increase in the individual's perception of excited level, and the individual is male. In some embodiments of the nasal delivery devices, the effect on the individual's mood includes an increase in the individual's perception of energetic level, and the individual is male.

In some embodiments of the nasal delivery devices, the effect on the individual's mood includes an increase in the individual's sensuality.

In some embodiments of the nasal delivery devices, the effect on the individual's mood includes an increase in the individual's personal well-being, and the individual is female.

In some embodiments of the nasal delivery devices, the nasal delivery device further includes an odorant.

In yet another aspect of the invention are provided nozzled bottles containing at least one 20-Pregnene compound having the formula:

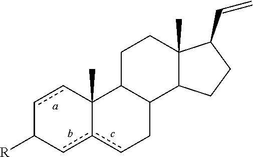

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond; and, wherein the at least one 20-pregenene compound is present in a non-therapeutic formulation.

In some embodiments, of the nozzled bottles, the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one. In some embodiments, the nozzled bottle is selected from the group consisting of atomizers, spray bottles, aerosol bottles and aerosol cans. In some embodiments, the 20-pregnene compound is formulated for delivery as an aerosol. In some embodiments, the 20-pregnene compound is formulated for delivery as a spray. In some embodiments, the 20-pregnene compound is formulated as 20-pregnene-containing composition selected from the group consisting of fragrance compositions comprising an odorant, personal care products, cleaning products, environmental products, and laundry products, wherein said 20-pregnene containing composition is suitable for delivery via a nozzled bottle.

In another aspect are provided kits containing packaging and at least one 20-Pregnene compound having the formula:

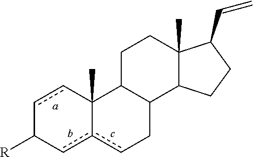

wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond; and wherein the at least one 20-pregenene compound is present in a non-therapeutic formulation.

In some embodiments of the kits the at least one 20-Pregnene compound occurs naturally in at least one of the following species of coral: Gersemia rubiformis, Carijoa riisei, Pieterfaurea unilobata, or Muricea fruticosa. In some embodiments, the at least one compound is selected from the group consisting of: pregna-5,20-dien-3β-ol and pregna-1,4,20-trien-3-one. In some embodiments, the at least one compound is pregna-5,20-dien-3β-ol. In some embodiments, the at least one compound is pregna-1,4,20-trien-3-one. In some embodiments, the packing comprises a nozzled bottle, sealed bottle, canister, paper wrapping, plastic wrapping, or box. In some embodiments, the at least one 20-pregene compound is present as a 20-pregnene-containing composition selected from the group consisting of fragrance compositions comprising an odorant, personal care products, cleaning products, environmental products, compositions comprising a fibrous material or polymeric component, and laundry products. In some embodiments, the packaging comprises a nozzled bottle. In some embodiments, the packaging comprises a nasal delivery device.

In another aspect are provided compounds, 20-pregene-containing compositions, nasal delivery devices, nozzled bottles and kits for use in affecting the mood of an individual, wherein the wherein the effect on the individual's mood comprises at least one of the following: an increase in the individual's social warmth and friendliness, an increase in the individual's arousal/excitement, or an increase in the individual's sensuality. In certain embodiments, the compounds are present in non-therapeutic formulations.

### BRIEF DESCRIPTION OF THE DRAWINGS

FIG. 1 schematically illustrates the synthesis of Pregna-5,20-dien-3β-ol and Pregna-1,4,20-trien-3-one.

FIG. 2 schematically illustrates the synthesis of Pregn-20-en-3-ol, Pregn-20-en-3-yl Acetate and Pregna-1,20-dien-3-one.

FIG. 3 shows the effect of similar quantities (approximately 30 picograms) of androstadienone (ADO-1) and muricin aglycone (1) on the induction of robust depolarization of the electrogram of nasal chemosensory receptors. Repeated stimulation with muricin aglycone (1, 2, 3) produces fatigue of the receptors as shown by the electrogram induced by subsequently delivered androstadienone (ADO-2).

FIG. 4 is a schematic diagram of the experimental design.

FIG. 5 shows the effect of nasal delivery of 12 ng pregna-5,20-dien-3β-ol versus control on the Sensuality cluster in men and women, as measured by the modified Derogatis Affective Balance Score (DABS)-70 Inventory.

FIG. 6 shows the effect of nasal delivery of 12 ng pregna-5,20-dien-3β-ol versus control on the Arousal/Excitement cluster in men and women, as measured by the modified DABS-70 Inventory.

FIG. 7 shows the effect of nasal delivery of 12 ng pregna-5,20-dien-3β-ol versus control on the Social Warmth and Friendliness cluster in men and women, as measured by the modified DABS-70 Inventory.

FIG. 8 shows the effect of nasal delivery of 12 ng pregna-5,20-dien-3β-ol versus control on the Personal Well-Being cluster in men and women, as measured by the modified DABS-70 Inventory.

FIG. 9 shows the effect of nasal delivery of 12 ng pregna-5,20-dien-3β-ol versus control on the Negativity cluster in men and women, as measured by the modified DABS-70 Inventory.

FIG. 10 shows the effect of nasal delivery of 12 ng pregna-5,20-dien-3β-ol (filled bars) versus control (open bars) on physiologic parameters in men and women. The “*” indicates statistically significant differences at 95% confidence intervals.

FIG. 11 shows the effect of nasal delivery of 250 picograms pregna-1,4,20-trien-3-onel versus control on physiologic parameters in men and women (A: skin temperature (ST), galvanic skin response (GSR), local electrogram of nasal chemoreceptors; B: EEG for midline frontal-occipital cortex (alpha frequency band) (EEG-v(a)), EEG for midline frontal-occipital cortex (beta frequency band) (EEG-v(b)), EEG for temporal cortex (alpha frequency band) (EEG-t(a)), EEG for temporal cortex (beta frequency band) (EEG-t(b)).

### DETAILED DESCRIPTION

Provided herein are 20-pregnene compounds that have an affect on human mood, as well as compositions containing and methods of using such compounds. It is surprising that the compounds described herein produce a particular psychological effect when presented intranasally, despite said compounds being odorless. Surprisingly, the psychological effects produced by the compounds described herein may be markedly different from those produced by androstadienone. For example, the psychological effects produced by pregna-5,20-dien-3β-ol (also referred to as muricin aglycone) were entirely different from those produced by androstadienone (which decreased negativity but had no effect on social warmth and friendliness, personal well-being and arousal/excitement), and were unexpected and surprising, particularly in view of the similarity of the effects of these two compounds on peripheral nasal chemosensory receptors (see FIG. 3 and Grosser et al., 2000).

The compounds, and compositions comprising the compounds, of the invention may also surprisingly similarly affect one or more categories of mood in both men and women, such as in the categories of social warmth and friendliness, arousal/excitement, and/or sensuality. The compounds of the invention may further have fixative qualities.

### DEFINITIONS

As used herein, “α-hydroxy” indicates —OH below the plane of the two-dimensional representation of the molecule.

As used herein, “β-hydroxy” indicates —OH above the plane of the two-dimensional representation of the molecule.

As used herein, “α-acetoxy” indicates —OC(═O)CH₃ below the plane of the two-dimensional representation of the molecule.

As used herein, “β-acetoxy” indicates —OC(═O)CH₃ above the plane of the two-dimensional representation of the molecule.

As used herein, “lower α-acyloxy” indicates —OC(═O)-lower alkyl below the plane of the two-dimensional representation of the molecule, wherein lower alkyl is C₁-C₆ alkyl (e.g., C₁, C₂, C₃, C₄, C₅, or C₆) and includes straight, branched, and cyclic alkyl.

As used herein, “lower β-acyloxy” indicates —OC(═O)-lower alkyl above the plane of the two-dimensional representation of the molecule, wherein lower alkyl is C1-C6 alkyl, and includes straight, branched, and cyclic alkyl.

As used herein, “social warmth and friendliness” indicates the category containing the cluster of psychometric measures of the feelings: sympathetic, sentimental, compassionate, loving, gentle, friendly, affectionate, and warm, as measured by the modified DABS-70 Inventory described herein.

As used herein, “loving level” indicates the level of the feeling: loving, as measured by the modified DABS-70 Inventory described herein.

As used herein, “warmth” indicates the level of the feeling: warm, as measured by the modified DABS-70 Inventory described herein.

As used herein, “arousal/excitement” indicates the category containing the cluster of psychometric measures of the feelings: vigorous, excited, energetic, active, lively, decisive, independent, adventurous, strong, dominant, assertive, and bold, as measured by the modified DABS-70 Inventory described herein.

As used herein, “excited level” indicates the level of the feeling: excited, as measured by the modified DABS-70 Inventory described herein.

As used herein, “energetic” indicates the level of the feeling: energetic, as measured by the modified DABS-70 Inventory described herein.

As used herein, “sensuality” indicates the category containing the cluster of psychometric measures of the feelings: feline, flirtatious, seductive, and passionate, as measured by the modified DABS-70 Inventory described herein.

As used herein, “personal well-being” indicates the category containing the cluster of psychometric measures of the feelings: confident, calm, satisfied, relaxed, contented, and pleased, as measured by the modified DABS-70 Inventory described herein.

As used herein, “negativity” indicates the category containing the cluster of psychometric measures of the feelings: sensitive, nervous, regretful, blameworthy, tense, ashamed, anxious, guilty, remorseful, sad, irritable, hopeless, resentful, worthless, angry, miserable, enraged, unhappy, bitter, and timid, as measured by the modified DABS-70 Inventory described herein.

### 20-Pregnene Compounds

The 20-pregnene compounds of the invention are selected from compounds having the formula:

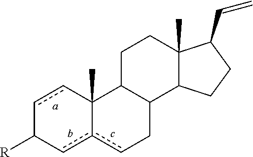

wherein R is selected from the group consisting of: oxo, α-hydroxy, β-hydroxy, α-acetoxy, β-acetoxy, lower α-acyloxy, and lower β-acyloxy; and wherein a, b, and c are individually optional double bonds, with the proviso that b and c are not both double bonds, and with the further proviso that if R is oxo then a is a double bond. In some embodiments, R is oxo. In some embodiments, R is α-hydroxy. In some embodiments, R is β-hydroxy. In some embodiments, R is α-acetoxy. In some embodiments, R is β-acetoxy. In some embodiments, R is lower α-acyloxy. In some embodiments, R is lower β-acyloxy. In some embodiments, a is a double bond. In some embodiments, a is a single bond. In some embodiments, b is a double bond. In some embodiments, b is a single bond. In some embodiments, c is a double bond. In some embodiments, c is a single bond. In some embodiments, a and b are double bonds. In some embodiments, a and c are double bonds. In some embodiments, a, b, and c are single bonds. In some embodiments, R is in the configuration (below the plane of the two dimensional representation of the compound), b is a single bond and the hydrogen at the 5 position of the molecules is also in the α configuration. In some embodiments, the 20-Pregnene compounds occur naturally in at least one of the following species of coral: Gersemia rubiformis, Carijoa riisei, Pieterfaurea unilobata, Muricea fruticosa, or the soft coral described in Higgs and Faulkner, Steroids, 1977, 30(3), 379. In some embodiments the compound is pregna-5,20-dien-3β-ol, pregna-1,4,20-trien-3-one, 5α-pregn-20-en-3α-ol, or 5α-pregn-20-en-3-yl-acetate. In some embodiments the compound is pregna-5,20-dien-3β-ol, pregna-1,4,20-trien-3-one, 5α-pregn-20-en-3α-ol, 5α-pregn-20-en-3β-yl-acetate, 5α-pregn-20-en-3β-ol, 5α-pregn-20-en-3β-yl acetate or 5α-pregna-1,20-dien-3-one. In some embodiments, the compound is pregna-5,20-dien-3β-ol. In some embodiments, the compound is pregna-1,4,20-trien-3-one. In some embodiments, the compound is 5α-pregn-20-en-3β-ol. In some embodiments, the compound is 5α-pregn-20-en-3α-yl acetate. In some embodiments, the compound is 5α-pregna-1,20-dien-3-one. In some embodiments, the compound is 5α-pregn-20-en-3α-ol. In some embodiments, the compound is a 20-pregnene compound other than pregna-5,20-dien-3β-ol. In some embodiments the compound is not pregna-5,20-dien-3β-ol, or pregna-1,4,20-trien-3-one. In some embodiments the compound is 5α-pregn-20-en-3α-ol, 5α-pregn-20-en-3β-yl-acetate, 5α-pregn-20-en-3β-ol, 5α-pregn-20-en-3α-yl acetate or 5α-pregna-1,20-dien-3-one. The compositions of the invention may contain 1, 2, 3, 4, 5, 10, or more 20-pregnene compounds as defined herein. The methods of the invention may utilize 1, 2, 3, 4, 5, 10, or more 20-pregnene compounds as defined herein. In some embodiments, the composition contains a single 20-pregnene compound as defined herein. In some embodiments, the composition contains two or more 20-pregnene compounds as defined herein. In some embodiments, the method comprises delivering a single 20-pregnene compound as defined herein. In some embodiments, the method comprises delivering two or more 20-pregnene compounds as defined herein.

### Methods of Affecting Moods

The 20-pregnene compounds described herein may be used to affect the mood of individuals. In some embodiments, the individual is a mammal. In some embodiments, the individual is a human. In some embodiments, the individual is female. In some embodiments, the individual is male. In some embodiments, the individual is a human female. In some embodiments, the individual is a human male. In some embodiments, the effect on the individual's mood comprises an increase in the individual's social warmth and friendliness. In some embodiments, the effect on the individual's mood comprises an increase in the individual's arousal/excitement. In some embodiments, the effect on the individual's mood comprises an increase in the individual's sensuality. The effect of the compound on mood may be similar for both males and females. For example, the compound may increase the individual's social warmth and friendliness, arousal/excitement, and/or sensuality in both males and females. In some embodiments, the effect on the individual's mood comprises an increase in the individual's perception of loving level, and wherein the individual is female. In some embodiments, the effect on the individual's mood comprises an increase in the individual's perception of warmth level, and wherein the individual is female. In some embodiments, the effect on the individual's mood comprises an increase in the individual's perception of arousal/excitement, and wherein the individual is female. In some embodiments, the effect on the individual's mood comprises an increase in the individual's perception of arousal/excitement, and wherein the individual is male. In some embodiments, the effect on the individual's mood comprises an increase in the individual's perception of excited level, and wherein the individual is female. In some embodiments, the effect on the individual's mood comprises an increase in the individual's perception of excited level, and wherein the individual is male. In some embodiments, the effect on the individual's mood comprises an increase in the individual's perception of energetic level, and wherein the individual is male. The compound(s) may further alter the individual's autonomic nervous system functions (such as heart rate, body temperature, etc.), brain waves, blinking rate, etc.

The method may comprise delivering the one or more compounds to the individual by nasal administration. The method may comprise delivering at least about 50 picograms, at least about 100 picograms, at least 250 picograms, at least about 300 picograms, at least about 500 picograms, at least about 1000 picograms, at least about 1 nanogram, at least about 10 nanograms, at least about 100 nanograms, at least about 1 microgram, at least about 10 micrograms of the at least one compound to the individual. In some embodiments, the method comprises delivering about 50 picograms to about 100 nanograms of the at least one compound to the individual. In some embodiments, the method comprises delivering about 50 picograms to about 10 micrograms. In some embodiments, the method comprises delivering about 50 picograms to about 10 nanograms. In some embodiments, the method comprises delivering about 50 picograms to about 1 nanogram. In some embodiments of the methods include delivering about 250 picograms to about 100 nanograms. In some embodiments of the methods include delivering about 300 picograms to about 100 nanograms. In some embodiments of the methods include delivering about 500 picograms to about 100 nanograms. In some embodiments, the amount delivered of the one or more compounds in the individual delivered intranasally is about 10 picograms to about 20 nanograms.

The one or more compounds may be delivered to the individual for at least about 10 seconds, at least about 30 seconds, at least about 1 minute, at least about 2 minutes, at least about 5 minutes, at least about 10 minutes, at least about 15 minutes, at least about 20 minutes, at least about 25 minutes, at least about 30 minutes, at least about 45 minutes, at least about 1 hour, at least about 2 hours, at least about 3 hours, at least about 5 hours, at least about 6 hours, at least about 8 hours, at least about 10 hours, at least about 12 hours, at least about 18 hours. In some embodiments, the compound is delivered to the individual over a time period of about 10 seconds to about 18 hours. In some embodiments, the compound is delivered to the individual over a time period of about 30 seconds to about 12 hours. In some embodiments, the compound is delivered to the individual over a time period of about 30 seconds to about 12 hours. In some embodiments, the compound is delivered to the individual over a time period of about 60 seconds to about 8 hours. In some embodiments, the compound is delivered to the individual over a time period of about 1 minute to about 2 hours. In some embodiments, the compound is delivered to the individual over a time period of about 1 minute to about 30 minutes. In some embodiments, the compound is delivered to the individual over a time period of about 12 minutes to about 18 hours. In some embodiments, the compound is delivered to the individual over a time period of about 15 minutes to about 18 hours. In some embodiments, the compound is delivered to the individual over a time period of about 20 minutes to about 18 hours.

The methods may comprise delivering to the individual at least about 500 picograms of the at least one compound over a time period of at least about 1 second. In some embodiments, at least about 250 picograms of the compound is delivered to the individual over a time period of at least about 1 second. In some embodiments, at least about 1 nanogram of the compound is delivered to the individual over a time period of at least about 1 second. In some embodiments, at least about 500 picograms of the compound is delivered to the individual over a time period of at least about 10 minutes. In some embodiments, at least about 1 nanograms of the compound is delivered to the individual over a time period of at least about 10 minutes. In some embodiments, at least about 10 nanograms of the compound is delivered to the individual over a time period of at least about 10 minutes. The methods may comprise delivering to the individual at least about 100 picograms of the at least one compound over a time period of at least about 1 minute. In some embodiments, at least about 250 picograms of the compound is delivered to the individual over a time period of at least about 1 minute. In some embodiments, at least about 500 picograms of the compound is delivered to the individual over a time period of at least about 1 minute. In some embodiments, at least about 1 nanograms of the compound is delivered to the individual over a time period of at least about 1 minute. In some embodiments, at least about 10 nanograms of the compound is delivered to the individual over a time period of at least about 1 minute. In some embodiments, at least about 15 nanograms of the compound is delivered to the individual over a time period of at least about 1 minute. In some embodiments, the at least one compound is administered continuously over the time period.

The methods may comprise delivering to the individual at least about 250 picograms of the at least one compound per second. In some embodiments, at least about 500 picograms of the at least one compound is delivered to the individual per second. In some embodiments, at least about 1 nanogram of the at least one compound is delivered to the individual per second. In some embodiments, the at least one compound is administered continuously over the time period of administration.

The methods may deliver the at least one compound by the fragrance compositions, personal care products and/or home consumer products described herein. The method may also comprise delivering the at least one compound to the individual by a nasal inhaler. The method may also comprise delivering the at least one compound to the individual as a part of aromatherapy (e.g., combined with or in conjunction with incense, etc.).

### Compositions

The 20-pregnene compounds described herein may be formulated into various compositions, such as fragrance compositions (e.g., a perfume, a cologne, an eau de toilette, etc.), personal care products (e.g., a cosmetic, a hair care product, a bath or shower product, a skin care product, an antiperspirant, a deodorant, a massage oil, nail product, personal lubricant, etc.), unscented products (e.g., such as a product sprayed or otherwise applied to the skin), and home consumer products (e.g., a fibrous material, a laundry product, cleaning product, or an environmental product). These compositions may be non-therapeutic (e.g., not intended to treat (cure, ameliorate, slow progression) or prevent diseases (e.g., life threatening or life-altering diseases (e.g., autoimmune diseases (e.g., rheumatoid arthritis, lupus, etc.), infection, cancer, seizures (e.g., as associated with epilepsy), etc.) or other conditions (e.g., pain (e.g., chronic, acute (e.g., as the result of injury, etc.), insomnia, pre-menstrual syndrome, etc.)) depression (e.g., postnatal depression, etc.), anxiety, etc.), or over-the-counter (OTC), or non-prescription therapeutic (e.g., formulated with an OTC or non-prescription active ingredient (e.g., retinoic acid, anti-acne ingredients, OTC or non-prescription anti-allergy (e.g., hydrocortisone, etc.), OTC or non-prescription anti-infectives (e.g., topical anti-bacterials, etc.) intended to treat or prevent a disease or condition). In some embodiments, the compositions are non-therapeutic. The compositions may be formulated to deliver the at least one 20-pregene compound in a manner consistent with the methods described herein (e.g., formulated to deliver the amount of at least one 20-pregnene compound as described herein to achieve the effects described herein (e.g., amount of compound delivered to an individual, amount of compound delivered per unit time, time period for exposure to 20-pregenen compounds, etc. to achieve the effect of on an individual's mood including at least one of the following: an increase in the individual's social warmth and friendliness, an increase in the individual's arousal/excitement, or an increase in the individual's sensuality). In general, the one or more 20-pregnene compounds may be present in the fragrance, personal care product, and home consumer product compositions at a total concentration of, for example, at least about 0.001%. For example, at least about 0.001%, at least about 0.01%, at least about 0.1%, at least about 0.5%, at least about 1%, at least about 2%, at least about 3%, at least about 4%, at least about 5%. In some embodiments, the one or more 20-pregnene compounds may be present in the composition at a total concentration of about 0.001%, about 0.01%, about 0.1%, at 0.5%, about 1%, about 2%, about 3%, about 4%, about 5%. In some embodiments the one or more 20-pregnene compounds may be present in the composition at a total concentration of less than 0.1%. In some embodiments, the one or more 20-pregnene compounds may be present in the composition at a total concentration of about 0.001% to about 5%, about 1% to about 2%, about 1% to about 3%, about 0.01% to about 5%, about 0.1% to about 5%. In some embodiments, the one or more 20-pregnene compounds may be present in the composition at a total concentration of about 100 ng/ml to about 1 mg/ml. In some embodiments, the one or more 20-pregnene compounds may be present in the composition at a total concentration of about 1 μg/ml to about 100 μg/ml. In some embodiments, the one or more 20-pregnene compounds may be present in the composition at a total concentration of about 1 ng/ml to about 50 mg/ml. In some embodiments, the one or more 20-pregnene compounds may be present in the composition at a total concentration of about 1 ng/ml to about 40 mg/ml, about 1 ng/ml to about 30 mg/ml, about 1 ng/ml to about 20 mg/ml, about 1 ng/ml to about 10 mg/ml, about 1 ng/ml to about 1 mg/ml, about 100 ng/ml to about 10 mg/ml, about 100 ng/ml to about 1 mg/ml, about 100 ng/ml to about 1 mg/ml, about 100 ng/ml to about 100 μg/ml, about 100 ng/ml to about 10 μg/ml, about 100 ng/ml to about 1 μg/ml. The concentration of 20-pregnene compound(s) may vary according to the particular compound(s) used, the type of composition, the amount of composition typically used by the individual, the effect of other composition ingredients on the release rate of the 20-pregnene compound(s), and the like. In some embodiments, the at least one compound is stable under the influence of light, heat, air, and moisture. In some embodiments, strong oxidizing agents are not present in the compositions.

As noted above, it is intended that the compositions described herein may be used in accordance with the methods described herein.

### Fragrance Compositions

Fragrance compositions contain at least one odorant. In some embodiments, the odorant is pleasant. Non-limiting examples of odorants include lavender, rose oil, vanillin, etc. The total amount of all odorants in the composition may be, for example, at least about 1%. In some embodiments, the total amount of all odorants comprises about 12% by weight of the composition. In some embodiments, the total amount of all odorants comprises about 5% by weight of the composition. In some embodiments, the total amount of all odorants comprises about 1% to about 30% by weight of the composition. In some embodiments, the total amount of all odorants comprises about 1% to about 20% by weight of the composition. The fragrance composition may be, for example, a perfume, cologne, eau de toilette, mist or perfumed wax. The fragrance composition may be formulated for external application to the skin.

The one or more compounds may be present in the fragrance composition at a concentration of, for example, at least about 1%. The one or more compounds may be present in the fragrance composition at a concentration of, for example, about 100 ng/ml to about 1 mg/ml, about 1 μg/ml to about 100 μg/ml, about 10 μg/ml. The one or more compounds may be present in the fragrance composition at a concentration of at least about 0.001%. For example, at least about 0.001%, at least about 0.01%, at least about 0.1%, at least about 0.5%, at least about 1%, at least about 2%, at least about 3%, at least about 4%, at least about 5%. In some embodiments, the one or more 20-pregnene compounds may be present in the fragrance composition at a total concentration of about 0.001%, about 0.01%, about 0.1%, at 0.5%, about 1%, about 2%, about 3%, about 4%, about 5%. In some embodiments the one or more 20-pregnene compounds may be present in the fragrance composition at a total concentration of less than 0.1%. In some embodiments, the one or more 20-pregnene compounds may be present in the fragrance composition at a total concentration of about 0.001% to about 5%, about 1% to about 2%, about 1% to about 3%, about 0.01% to about 5%, about 0.1% to about 5%. In some embodiments, the one or more 20-pregnene compounds may be present in the fragrance composition at a total concentration of about 100 ng/ml to about 1 mg/ml. In some embodiments, the one or more 20-pregnene compounds may be present in the fragrance composition at a total concentration of about 1 μg/ml to about 100 μg/ml. In some embodiments, the one or more 20-pregnene compounds may be present in the fragrance composition at a total concentration of about 1 ng/ml to about 50 mg/ml. In some embodiments, the one or more 20-pregnene compounds may be present in the fragrance composition at a total concentration of about 1 ng/ml to about 40 mg/ml, about 1 ng/ml to about 30 mg/ml, about 1 ng/ml to about 20 mg/ml, about 1 ng/ml to about 10 mg/ml, about 1 ng/ml to about 1 mg/ml, about 100 ng/ml to about 10 mg/ml, about 100 ng/ml to about 1 mg/ml, about 100 ng/ml to about 1 mg/ml, about 100 ng/ml to about 100 μg/ml, about 100 ng/ml to about 10 μg/ml, about 100 ng/ml to about 1 μg/ml.

### Personal Care Product Compositions

The personal care product may be, for example, a cosmetic, a hair care product, a bath or shower product, a skin care product, an antiperspirant, a deodorant, a massage oil, a nail product (e.g., nail varnish, nail strengthener, nail sealer, etc.), or a personal lubricant. Cosmetic compositions include, for example, lipstick, lip gloss, concealer, mascara, face powder, foundation, eye shadow, eyeliner, nail products, etc. Cosmetics may be formulated for application to the skin of the face, the body, the eyelids, or lips; or the eyelashes; or nails. Cosmetics may additionally include, for example, solvents, oils, waxes, etc. For example, aloe, propylene glycol, dimethicone, etc. Hair care products include, for example, shampoos, conditioners, hair gels, hair treatment products, hair dyes, pomades, and hairsprays. Bath or shower products include, for example, soaps, non-soap cleansers, gels, foams, and lotions. Skin care products include, for example, moisturizers, shaving creams, shaving lotions, shaving gels, aftershave lotions, aftershave creams, aftershave balms, skin bronzers, skin bleaches, suncsreens, and topical analgesics. Skin care products may include OTC or non-prescription dermatological products that include one or more active ingredients intended to treat or prevent a disease or condition (e.g., retinoic acid, hydrocortisone, topical analgesic(s), anti-acne medication, anti-bacterials, cleansing lotions, etc.). Personal care products include, for example, masculine and feminine lubricants, which may be pre-applied to a condom. In some embodiments, the personal care product may be non-therapeutic (e.g., not intended to treat (cure, ameliorate, slow progression) or prevent diseases or other conditions).

The personal care product may further comprise at least one odorant. In some embodiments, the odorant is pleasant. Non-limiting examples of odorants include lavender, rose oil, vanillin, etc. The total amount of all odorants in the composition may be, for example, at least about 1%. In some embodiments, the total amount of all odorants comprises about 5% by weight of the composition. In some embodiments, the total amount of all odorants comprises about 1% to about 30% by weight of the composition. In some embodiments, the total amount of all odorants comprises about 1% to about 20% by weight of the composition.

The personal care product composition may be formulated for external application to the skin, hair, nails or eyelashes. The one or more 20-pregnene compounds may be present in the personal care product at a concentration of, for example, at least about 1%. The one or more compounds may be present in the personal care product at a concentration of, for example, about 100 ng/ml to about 1 mg/ml, about 1 μg/ml to about 100 μg/ml, about 10 μg/ml. The one or more compounds may be present in the personal care product at a concentration of at least about 0.001%. For example, at least about 0.001%, at least about 0.01%, at least about 0.1%, at least about 0.5%, at least about 1%, at least about 2%, at least about 3%, at least about 4%, at least about 5%. In some embodiments, the one or more 20-pregnene compounds may be present in the personal care product at a total concentration of about 0.001%, about 0.01%, about 0.1%, at 0.5%, about 1%, about 2%, about 3%, about 4%, about 5%. In some embodiments the one or more 20-pregnene compounds may be present in the personal care product at a total concentration of less than 0.1%. In some embodiments, the one or more 20-pregnene compounds may be present in the personal care product at a total concentration of about 0.001% to about 5%, about 1% to about 2%, about 1% to about 3%, about 0.01% to about 5%, about 0.1% to about 5%. In some embodiments, the one or more 20-pregnene compounds may be present in the personal care product at a total concentration of about 100 ng/ml to about 1 mg/ml. In some embodiments, the one or more 20-pregnene compounds may be present in the personal care product at a total concentration of about 1 μg/ml to about 100 μg/ml. In some embodiments, the one or more 20-pregnene compounds may be present in the personal care product at a total concentration of about 1 ng/ml to about 50 mg/ml. In some embodiments, the one or more 20-pregnene compounds may be present in the personal care product at a total concentration of about 1 ng/ml to about 40 mg/ml, about 1 ng/ml to about 30 mg/ml, about 1 ng/ml to about 20 mg/ml, about 1 ng/ml to about 10 mg/ml, about 1 ng/ml to about 1 mg/ml, about 100 ng/ml to about 10 mg/ml, about 100 ng/ml to about 1 mg/ml, about 100 ng/ml to about 1 mg/ml, about 100 ng/ml to about 100 μg/ml, about 100 ng/ml to about 10 μg/ml, about 100 ng/ml to about 1 μg/ml.

### Home Consumer Product Compositions

The home consumer product may be, for example, a fibrous material, a polymeric material, a laundry product, a cleaning product, or an environmental product. Fibrous materials include, for example, fabric and paper products (e.g., tissues, wipes, etc.). The tissues may further comprise one or more additional components, such as, for example, moisturizer, odorants, etc. The wipes may further comprise one or more additional components, such as, for example, moisturizerpioc, skin sanitizers, skin balms, topical analgesics, skin bronzer, skin lightening agents, suncreens, etc., agents to enhance evaporation and/or dispersal of the 20-pregnane compounds, odorants, etc. The home consumer product may also include tapes, elastic fabric supports (e.g., athletic supports, etc.) and wound coverings, which may include fibrous components (e.g., bandages, surgical tapes, etc.) and/or polymeric components (e.g., polymer-based adhesive wound coverings, surgical tapes, etc). Polymeric components may be polymers that occur in nature or synthetic polymers, not occurring in nature. The polymeric components may also include an adhesive component. Laundry products include, for example, liquid or powdered laundry detergents, liquid or powdered laundry soaps, bleaches, stain removers, and fabric softeners. Cleaning products include, for example, dishwashing soaps or detergents (e.g., for hand or automatic dishwashing), disinfectants, sterilizers, floor cleaners (e.g., for wood, linoleum, vinyl, carpet), appliance cleaners (e.g., for stainless steel, enamel, etc.), glass cleaners, tile cleaners, laminate cleaners, stone cleaners (e.g., granite, marble, soapstone, etc.), etc. Environmental products include, for example, room fresheners, car fresheners, and candles. In some embodiments, the home consumer product may be non-therapeutic (e.g., not intended to treat (cure, ameliorate, slow progression) or prevent diseases or other conditions).

The home consumer product may further comprise at least one odorant. In some embodiments, the odorant is pleasant. Non-limiting examples of odorants include lavender, rose oil, vanillin, etc. The total amount of all odorants in the composition may be, for example, at least about 1%. In some embodiments, the total amount of all odorants comprises about 5% by weight of the composition. In some embodiments, the total amount of all odorants comprises about 1% to about 30% by weight of the composition. In some embodiments, the total amount of all odorants comprises about 1% to about 20% by weight of the composition.

The one or more 20-pregnene compounds may be present in the home consumer product composition at a concentration of, for example, at least about 1%. The one or more compounds may be present in the home consumer product at a concentration of, for example, about 100 ng/ml to about 1 mg/ml, about 1 μg/ml to about 100 μg/ml, about 10 μg/ml. The one or more compounds may be present in the home consumer product at a concentration of at least about 0.001%. For example, at least about 0.001%, at least about 0.01%, at least about 0.1%, at least about 0.5%, at least about 1%, at least about 2%, at least about 3%, at least about 4%, at least about 5%. In some embodiments, the one or more 20-pregnene compounds may be present in the home consumer product at a total concentration of about 0.001%, about 0.01%, about 0.1%, at 0.5%, about 1%, about 2%, about 3%, about 4%, about 5%. In some embodiments the one or more 20-pregnene compounds may be present in the home consumer product at a total concentration of less than 0.1%. In some embodiments, the one or more 20-pregnene compounds may be present in the home consumer product at a total concentration of about 0.001% to about 5%, about 1% to about 2%, about 1% to about 3%, about 0.01% to about 5%, about 0.1% to about 5%. In some embodiments, the one or more 20-pregnene compounds may be present in the home consumer product at a total concentration of about 100 ng/ml to about 1 mg/ml. In some embodiments, the one or more 20-pregnene compounds may be present in the home consumer product at a total concentration of about 1 μg/ml to about 100 μg/ml. In some embodiments, the one or more 20-pregnene compounds may be present in the home consumer product at a total concentration of about 1 ng/ml to about 50 mg/ml. In some embodiments, the one or more 20-pregnene compounds may be present in the home consumer product at a total concentration of about 1 ng/ml to about 40 mg/ml, about 1 ng/ml to about 30 mg/ml, about 1 ng/ml to about 20 mg/ml, about 1 ng/ml to about 10 mg/ml, about 1 ng/ml to about 1 mg/ml, about 100 ng/ml to about 10 mg/ml, about 100 ng/ml to about 1 mg/ml, about 100 ng/ml to about 1 mg/ml, about 100 ng/ml to about 100 μg/ml, about 100 ng/ml to about 10 μg/ml, about 100 ng/ml to about 1 μg/ml.

### Nasal Delivery Compositions

In some embodiments the 20-pregnene compounds described herein may be formulated into various compositions suitable for direct delivery into the nasal cavity via a nasal delivery device. For example, delivery via a nasal inhaler or other device. Nasal inhaler and other nasal delivery devices are well known to the skilled artisan and may be fabricated from any suitable material(s), including, for example, glass, metal, polymers (e.g., plastics, etc.) or combinations of the foregoing. Nasal inhalers and other nasal delivery devices generally include an aperture for direct delivery of the composition from the nasal delivery device into the nasal cavity of the subject.

Thus in some embodiments are provided a nasal delivery device containing at least one of the 20-pregnene compound as described herein. The nasal delivery device containing the 20-pregnene compounds will be formulated for nasal delivery and may additionally include a carrier. Various carriers suitable for use in nasal delivery devices are also well known the skilled artisan and may be, for example, a solid (e.g., cotton, polymer(s), sponge, etc.), liquid or gel. The nasal delivery device may also contain additional components, for example, dispersants, etc.

The nasal delivery devices may contain the at lest one 20-pregnene compound formulated to deliver the at least one 20-pregene compound in a manner consistent with the methods described herein (e.g., formulated to deliver the amount of at least one 20-pregnene compound as described herein to achieve the effects described herein (e.g., amount of compound delivered to an individual, amount of compound delivered per unit time, time period for exposure to 20-pregenen compounds, etc. to achieve the effect of on an individual's mood including at least one of the following: an increase in the individual's social warmth and friendliness, an increase in the individual's arousal/excitement, or an increase in the individual's sensuality). The one or more 20-pregnene compounds may be present in the formulation contained in the nasal delivery device at a concentration of, for example, at least about 1%. The one or more compounds may be present in the formulation contained in the nasal delivery device at a concentration of, for example, about 100 ng/ml to about 1 mg/ml, about 1 μg/ml to about 100 μg/ml, about 10 μg/ml. The one or more compounds may be present in the formulation contained in the nasal delivery device at a concentration of at least about 0.001%. For example, at least about 0.001%, at least about 0.01%, at least about 0.1%, at least about 0.5%, at least about 1%, at least about 2%, at least about 3%, at least about 4%, at least about 5%. In some embodiments, the one or more 20-pregnene compounds may be present in the formulation contained in the nasal delivery device at a total concentration of about 0.001%, about 0.01%, about 0.1%, at 0.5%, about 1%, about 2%, about 3%, about 4%, about 5%. In some embodiments the one or more 20-pregnene compounds may be present in the formulation contained in the nasal delivery device at a total concentration of less than 0.1%. In some embodiments, the one or more 20-pregnene compounds may be present in the formulation contained in the nasal delivery device at a total concentration of about 0.001% to about 5%, about 1% to about 2%, about 1% to about 3%, about 0.01% to about 5%, about 0.1% to about 5%. In some embodiments, the one or more 20-pregnene compounds may be present in the formulation contained in the nasal delivery device at a total concentration of about 100 ng/ml to about 1 mg/ml. In some embodiments, the one or more 20-pregnene compounds may be present in the formulation contained in the nasal delivery device at a total concentration of about 1 μg/ml to about 100 μg/ml. In some embodiments, the one or more 20-pregnene compounds may be present in the formulation contained in the nasal delivery device at a total concentration of about 1 ng/ml to about 50 mg/ml. In some embodiments, the one or more 20-pregnene compounds may be present in the formulation contained in the nasal delivery device at a total concentration of about 1 ng/ml to about 40 mg/ml, about 1 ng/ml to about 30 mg/ml, about 1 ng/ml to about 20 mg/ml, about 1 ng/ml to about 10 mg/ml, about 1 ng/ml to about 1 mg/ml, about 100 ng/ml to about 10 mg/ml, about 100 ng/ml to about 1 mg/ml, about 100 ng/ml to about 1 mg/ml, about 100 ng/ml to about 100 μg/ml, about 100 ng/ml to about 10 μg/ml, about 100 ng/ml to about 1 μg/ml.

It is intended that the nasal delivery devices may be used in accordance with the methods described herein. In some embodiments, the nasal delivery devices may be designed to be non-therapeutic (e.g., not intended to treat (cure, ameliorate, slow progression) or prevent diseases or other conditions).

### Nozzled Bottles

In some embodiments the 20-pregnene compounds described herein may be contained in a nozzled bottle. Nozzled bottles are well known to the skilled artisan and may be fabricated from any suitable material(s), including, for example, glass, metal, ceramic, polymers (e.g., plastics, etc.) or combinations of the foregoing. Nozzled bottles generally include a storage reservoir (e.g. suitable to contain a liquid, gas, or gel, (e.g., pressurized or unpressurized) within which the at least one 20-pregene compound is present) and a nozzle. The nozzled bottles contain at least one 20-pregnene compound as described herein. In some embodiments, the nozzled bottles contain at least one 20-pregnene compound formulated as described herein (wherein the formulation is suitable for delivery via a nozzled bottle). In some embodiments, the 20-pregnene compound is formulated as 20-pregnene-containing composition such as described herein, e.g., as fragrance compositions comprising an odorant, personal care products, cleaning products, environmental products, and laundry products, wherein the 20-pregnene containing composition is suitable for delivery via a nozzled bottle. In some embodiments, the 20-pregnene-containing compositions may be non-therapeutic (e.g., not intended to treat (cure, ameliorate, slow progression) or prevent diseases or other conditions) (e.g., cleaning products, laundry products, etc.).

A wide variety of nozzled bottles will be well known by the skilled artisan, particularly in view of the teachings provided herein. Suitable nozzled bottles include, for example, atomizers, spray bottles, aerosol bottles, aerosol cans, etc. Suitable materials for fabrication of the nozzled bottles will also be well known by the skilled artisan and include, but are not limited to, e.g., glass, metal, polymers (e.g., plastics, etc.) or combinations of the foregoing. In some embodiments the at least one 20-pregnene compound is formulated for delivery as a spray (e.g. where “spray” refers to the conversion of bulk liquid into mist (i.e. collection of drops), by passing the liquid through a nozzle). Thus, in some embodiments, the 20-pregenene compounds are formulated for delivery as a spray and may be delivered by, for example a spray bottle (e.g., including pump bottles, trigger spray bottles, etc., such as those used to deliver personal care products (e.g., sunscreens, etc.), laundry products (e.g., stain removers, etc.), cleaning products (e.g., glass cleaners, disinfectants, etc.), etc.) or atomizer (e.g., such as those used to deliver fragrance compositions containing an odorant (e.g., perfumes, etc.), environmental products (e.g., room fresheners, etc.), etc.). In some embodiments the at least one 20-pregnene compound is formulated for delivery as an aerosol (e.g. where “aerosol” refers to a suspension of fine solid or liquid droplets in a gas that are delivered through the nozzle). Thus, in some embodiments, the 20-pregenene compounds are formulated for delivery as an aerosol and may be delivered by, for example an aerosol bottle or aerosol can (e.g., such as those used to deliver personal care products (e.g., sunscreens, deodorants, shaving foam, etc.), laundry products (e.g., stain removers, etc.), cleaning products (e.g., appliance cleaners, etc.), etc.) or atomizer (e.g., such as those used to deliver fragrance compositions containing an odorant (e.g., perfumes, etc.), environmental products (e.g., room fresheners, etc.), etc.).

Thus in some embodiments are provided a nozzled bottles containing at least one of the 20-pregnene compound as described herein. The nozzled bottles containing the 20-pregnene compounds may additionally include a carrier. Various carriers suitable for use in nozzled bottles are also well known the skilled artisan and may be, for example, a gas (e.g., propellant, etc.) liquid (e.g., water, alcohol (e.g., ethoanol) or gel. The nozzled bottle may also contain additional components, for example, dispersants, etc.

The nozzled bottle may contain the at lest one 20-pregnene compound formulated to deliver the at least one 20-pregene compound in a manner consistent with the methods described herein (e.g., formulated to deliver the amount of at least one 20-pregnene compound as described herein to achieve the effects described herein (e.g., amount of compound delivered to an individual, amount of compound delivered per unit time, time period for exposure to 20-pregenen compounds, etc. to achieve the effect of on an individual's mood including at least one of the following: an increase in the individual's social warmth and friendliness, an increase in the individual's arousal/excitement, or an increase in the individual's sensuality). The one or more 20-pregnene compounds may be present in the formulation contained in the nozzled bottle at a concentration of, for example, at least about 1%. The one or more compounds may be present in the formulation contained in the nozzled bottle at a concentration of, for example, about 100 ng/ml to about 1 mg/ml, about 1 μg/ml to about 100 μg/ml, about 10 μg/ml. The one or more compounds may be present in the formulation contained in the nozzled bottle at a concentration of at least about 0.001%. For example, at least about 0.001%, at least about 0.01%, at least about 0.1%, at least about 0.5%, at least about 1%, at least about 2%, at least about 3%, at least about 4%, at least about 5%. In some embodiments, the one or more 20-pregnene compounds may be present in the formulation contained in the nozzled bottle at a total concentration of about 0.001%, about 0.01%, about 0.1%, at 0.5%, about 1%, about 2%, about 3%, about 4%, about 5%. In some embodiments the one or more 20-pregnene compounds may be present in the formulation contained in the nozzled bottle at a total concentration of less than 0.1%. In some embodiments, the one or more 20-pregnene compounds may be present in the formulation contained in the nozzled bottle at a total concentration of about 0.001% to about 5%, about 1% to about 2%, about 1% to about 3%, about 0.01% to about 5%, about 0.1% to about 5%. In some embodiments, the one or more 20-pregnene compounds may be present in the formulation contained in the nozzled bottle at a total concentration of about 100 ng/ml to about 1 mg/ml. In some embodiments, the one or more 20-pregnene compounds may be present in the formulation contained in the nozzled bottle at a total concentration of about 1 μg/ml to about 100 μg/ml. In some embodiments, the one or more 20-pregnene compounds may be present in the formulation contained in the nozzled bottle at a total concentration of about 1 ng/ml to about 50 mg/ml. In some embodiments, the one or more 20-pregnene compounds may be present in the formulation contained in the nozzled bottle at a total concentration of about 1 ng/ml to about 40 mg/ml, about 1 ng/ml to about 30 mg/ml, about 1 ng/ml to about 20 mg/ml, about 1 ng/ml to about 10 mg/ml, about 1 ng/ml to about 1 mg/ml, about 100 ng/ml to about 10 mg/ml, about 100 ng/ml to about 1 mg/ml, about 100 ng/ml to about 1 mg/ml, about 100 ng/ml to about 100 μg/ml, about 100 ng/ml to about 10 μg/ml, about 100 ng/ml to about 1 μg/ml.

It is intended that the nozzled bottles may be used in accordance with the methods described herein.

### Kits

In some embodiments the 20-pregnene compounds described herein may be contained in a kit containing at least one 20-pregnene compound described herein and packaging. In some embodiments, the kits contain at least one 20-pregnene compound formulated as described herein. In some embodiments, the 20-pregnene compound is formulated as 20-pregnene-containing composition such as described herein, e.g., a fragrance compositions comprising an odorant, personal care products, cleaning products, environmental products, compositions comprising a fibrous material or polymeric component, and laundry products. In some embodiments the kits contain a nasal delivery device or nozzled bottle (e.g., spray bottle, aerosol bottle, aerosol can, etc.) as described herein. In some embodiments the kits contain a nasal delivery device. In some embodiments the kits contain a nozzled bottle. In some embodiments, the kits contain the compounds formulated in a non-therapeutic composition (e.g., not intended to treat (cure, ameliorate, slow progression) or prevent diseases or other conditions) (e.g., cleaning products, laundry products, etc.).

In some embodiments the packaging includes a nozzled bottle (such as described above and known to the skilled artisan), sealed bottle (e.g., a container with a neck that is narrower than the body and a narrower opening (e.g., glass, polymer (e.g., plastic), metal, ceramic, etc.) with a lid, stopper, or other covering for the narrower opening), e.g., screw top bottles, vials/ampoules, etc.), canisters (e.g., approximately cylindrical container), e.g., jars (e.g., glass, plastic or other impervious material), cardboard, metal, etc.), paper wrapping, plastic wrapping or box (e.g., cardboard, wooden, plastic, metal, etc.). Examples of suitable packaging will be known to the skilled artisan, particularly in view of the teaching provided herein. The skilled artisan will also be able to modify packaging to suit the particular 20-pregenene-containing composition being packaged. For example, the use of plastic bottles for liquid or gel laundry or dishwashing detergents versus the use of boxes for powdered laundry or dishwashing detergents, etc.

In some embodiments, the kits include instructions for use.

It is intended that the kits may be used in accordance with the methods described herein.

The invention is further illustrated by the following non-limiting examples.

### EXAMPLES

### Example 1

### Pregna-5,20-dien-3β-ol (3)

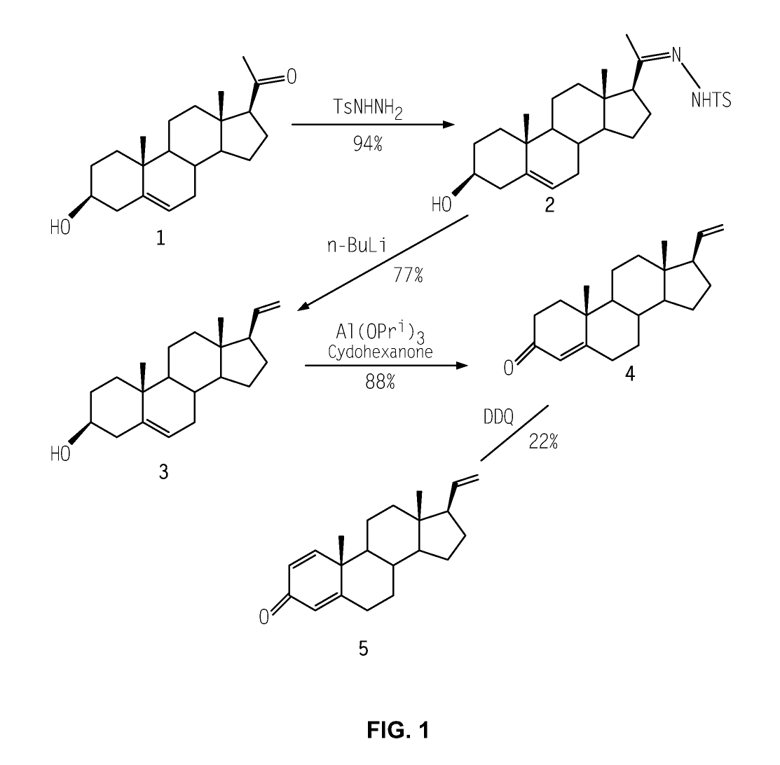

The synthesis of pregna-5,20-dien-3β-ol (3) is shown in FIG. 1. Pregnenolone (1) (15.8 g, 50.0 mmole) and p-toluenesulfonhydrazide (11.65 g, 62.5 mmole) in dry methanol (200 ml) were heated under reflux for 20 hours. The mixture was filtered while still hot, washing with methanol (100 ml). The solid was discarded, and the filtrate was evaporated in vacuo to 200 ml and allowed to cool to room temperature. The mixture was filtered, washing with methanol (20 ml). The crystals of the tosylhydrazone (2) were dried in vacuo to give 19.7 g. The filtrate was heated while being evaporated in vacuo to 50 ml and allowed to cool to room temperature. The mixture was filtered and the crystals of the tosylhydrazone were dried in vacuo to give 3.1 g. Total yield of the tosylhydrazone (2) was 22.8 g (94%).

To the tosylhydrazone (2) (12.1 g, 25.0 mmole) in dry THF (600 ml) cooled in ice/brine under argon was added n-BuLi (125 mmole, 50 ml of 2.5 M solution in hexane) and the mixture was stirred while allowing to warm to room temperature. After 48 hours the mixture was cooled in ice, and water (50 ml) was slowly added. The mixture was poured into chilled NH₄Cl solution and the resulting mixture was extracted with diethyl ether (x2). The ethereal extracts were washed with saturated NaHCO₃ solution and brine, dried (MgSO₄), and evaporated in vacuo to give 8.7 g crude material. This was recrystallized from methanol (40 ml), washing the crystals with a further 10 ml methanol. The crystals were dried in vacuo to give 5.8 g (77%) pregna-5,20-dien-3β-ol (3); m.p. 132-135° C. C₂₁M₃₂O calc. C, 83.94, H, 10.73. found C, 83.83, H, 10.50. JR (nujol): νₘₐₓ=3570 (hydroxy), 1665 cm⁻¹ (olefin); no carbonyl absorption. NMR (CDCl₃): δ=4.8-6.2 (4H, m, olefin), 1.02 (3H, s, 19-Me), 0.68 (3H, 18-Me).

### Example 2

### Pregna-1,4,20-trien-3-one (5)

The synthesis of pregna-1,4,20-triene-3-one (5) is shown in FIG. 1. A solution of pregna-5,20-dien-3β-ol (3) (6.00 g, 20.0 mmole) in toluene (285 ml) and cyclohexanone (45 ml) was distilled until 30 ml of distillate had been collected, which was then discarded. Aluminum isopropoxide (3.00 g) in toluene (30 ml) was added, and the mixture was heated under reflux for 1 hour. Water (500 ml) was then slowly added as the mixture was distilled until 500 ml of distillate had been collected. The distillate was discarded. A mixture of saturated sodium potassium tartrate solution (200 ml) and water (300 ml) was added, and the mixture was extracted with chloroform (x2). The combined extracts were dried (MgSO₄) and evaporated in vacuo to give 11.3 g crude material. This was purified by flash chromatography on 226 g silica gel (230-400 mesh), eluting with 10:90→20:80 EtOAc/hexane to give 5.25 g pregna-4,20-dien-3-one (4) (88%).

Pregna-4,20-dien-3-one (4) (1.19 g, 3.99 mmole) and 2,3-dichloro-5,6-dicyano-1,4-benzoquinone (DDQ) (2.72 g, 12.1 mmole) in benzene (40 ml) were heated under reflux under argon for 24 hours. The cooled suspension was diluted with diethyl ether and washed with two 100 ml portions of 5% (w/w) sodium hydroxide in water, two 100 ml portions of water, and once with 100 ml of brine. Diethyl ether (100 ml) was added to the resulting emulsion, which was dried over sodium sulfate and then filtered through a column of sodium sulfate (20 g). After washing the residue twice with 50 ml portions of diethyl ether, the combined filtrates were concentrated under reduced pressure and then purified by flash chromatography on silica gel (230-400 mesh), eluting with 25% EtOAc/hexane to give 0.26 g pregna-1,4,20-trien-3-one (5) (22%) as a slightly yellow crystalline solid.

### Example 3

### 5α-Pregna-1,20-dien-3-one (11, 5α)

The synthesis of 5α-pregna-1,20-dien-3-one is shown in FIG. 3 and can be performed as described in Schow and McMorris, Steroids, 1977, 30, 389, as reproduced below. A solution of 3β-hydroxy-5α-pregnan-20-one (6) (5.0 g, 0.016 mole, prepared by catalytic hydrogenation of 3β-hydroxy-5-pregnen-20-one with 10% Pd on charcoal), p-toluenesulfonylhydrazide (5 g, 0.027 mole) and concentrated hydrochloric acid (1 ml) in absolute ethanol (100 ml) is refluxed for two hours. The cooled reaction mixture is diluted with water (200 ml) and extracted with ether (4×150 ml). The combined extracts are washed with 10% hydrochloric acid (2×200 ml) and dried (MgSO₄). Removal of the solvent yielded 7.8 g (quantitative yield) of the unstable tosylhydrazone (7), mp 67-70° C.; νₘₐₓ 3420, 3200, 1600 cm⁻¹; δ 0.29 (s, C-18 Me), 0.77 (s; C-19 Me), 2.41 (s, C-21 Me), 3.54 (m, C-3αH), 7.30 (m, aromatic H), 7.86 (m, aromatic H) ppm. To a stirred solution of (7) (5.0 g, 0.01 mole) in anhydrous ether (75 ml) at 0° C. under argon is added dropwise a solution of n-butyl lithium (0.075 mole, 35.0 ml of a 2.1 molar solution in hexane) in anhydrous ether (45 ml). After addition is complete (approximately one hour), the reaction mixture is allowed to warm to room temperature and is stirred continuously for eight hours. Water (100 ml) is then slowly added and the aqueous solution extracted with ether (2×100 ml). The combined extracts are washed with water (200 ml), dried (MgSO₄) and the solvent removed. Chromatography of the residue on silica gel with petroleum ether (30-60° C.)-ethyl acetate gives 1.9 g (0.0063 mole, 61%) of (8). (8) is recrystallized from petroleum ether-chloroform, mp 130-132° C.; νₘₐₓ 3280, 1640 cm⁻¹; δ 0.58 (s, C-18 Me), 0.81 (s, C-19 Me), 3.58 (m, C-3αH), 4.88-4.99 (m, C-21H), 5.76 (m, C-20H); MS, m/e 302 (100%, M⁺), 287 (M-CH₃), 269 (M-CH₂—H₂O).

Jones reagent is added dropwise to a stirred solution of 5α-pregn-20-en-3β-ol (8) (1.2 g, 0.004 mole) in 150 ml of acetone at 0° C. Addition is stopped when the orange color persists. Water (200 ml) is added, and the product extracted with ether (3×150 ml). The combined extracts are washed with saturated sodium bicarbonate solution and then water, dried, and the ether removed, leaving 1.0 g (0.0033 mole, 83%) of the ketone (10). The ketone (10) is recrystallized from methanol, mp 115-117° C.; νₘₐₓ 1723, 1642 cm⁻¹; δ 0.61 (s, C-18 Me), 1.02 (s, C-19 Me), 4.81-5.02 (m, C-21H), 5.77 (m, C-20H); MS, m/e 300 (100%, M⁺), 285 (M-CH₃).

A solution of ketone (10) (0.15 g, 5.0×10⁻⁴ mole) in 1.5 ml of dry tetrahydrofuran is added to 0.70 ml of 0.80 molar lithium diisopropyl amide in tetrahydrofuran (5.6×10⁻⁴ mole) under argon at −78° C. After stiffing for one hour at −78° C., 0.21 g (0.0011 mole) of benzeneselenyl chloride in 1.5 ml of dry tetrahydrofuran was added quickly to the enolate solution. The reaction mixture is stirred for thirty minutes, then water (10 ml) was added and the mixture extracted with ether (4×20 ml). The combined extracts are washed with dilute hydrochloric acid (10%, 2×10 ml), saturated sodium bicarbonate solution (15 ml), water, dried, and the solvent removed. The crude product is dissolved in ethyl acetate (2 ml) and methanol (2 ml), and 0.5 g (0.0023 mole) of sodium metaperiodate dissolved in enough water-methanol-ethyl acetate to maintain solution is added. The reaction mixture is stirred for one hour, then water (20 ml) added and the mixture extracted with ether (4×15 ml). The combined extracts are dried and the solvent removed, leaving the product, which is purified by chromatography on silica gel (200 mesh with hexane-ethyl acetate, 100:5). This gives 0.082 g (2.8×10⁻⁴ mole, 55%) of the dienone (11), which on recrystallization from hexane-dichloromethane had mp 124-126° C. λₘₐₓ (CH₃OH) 228 nm (1.04×10⁴); νₘₐₓ 1692, 1648 cm⁻¹; δ 0.63 (s, C-18 Me), 1.02 (s, C-19 Me), 4.94-5.01 (m, C-21H), 5.78 (m, C-20H), 5.85 (d, J=10.2 Hz, C-2H), 7.16 (d, J=10.2 Hz, C-1H); MS, m/e 298.230 (68% M⁺; calcd. for C₂₁H₃₀O: 298.2298), 283 (M-15), 270 (M-CH₂CH₂), 122 (100% C₈H₁₀O); CD (θ) 337-470 (CH₃OH).

### Example 4

### 5α-Pregn-20-en-3α-yl Acetate (9, 3α5α)

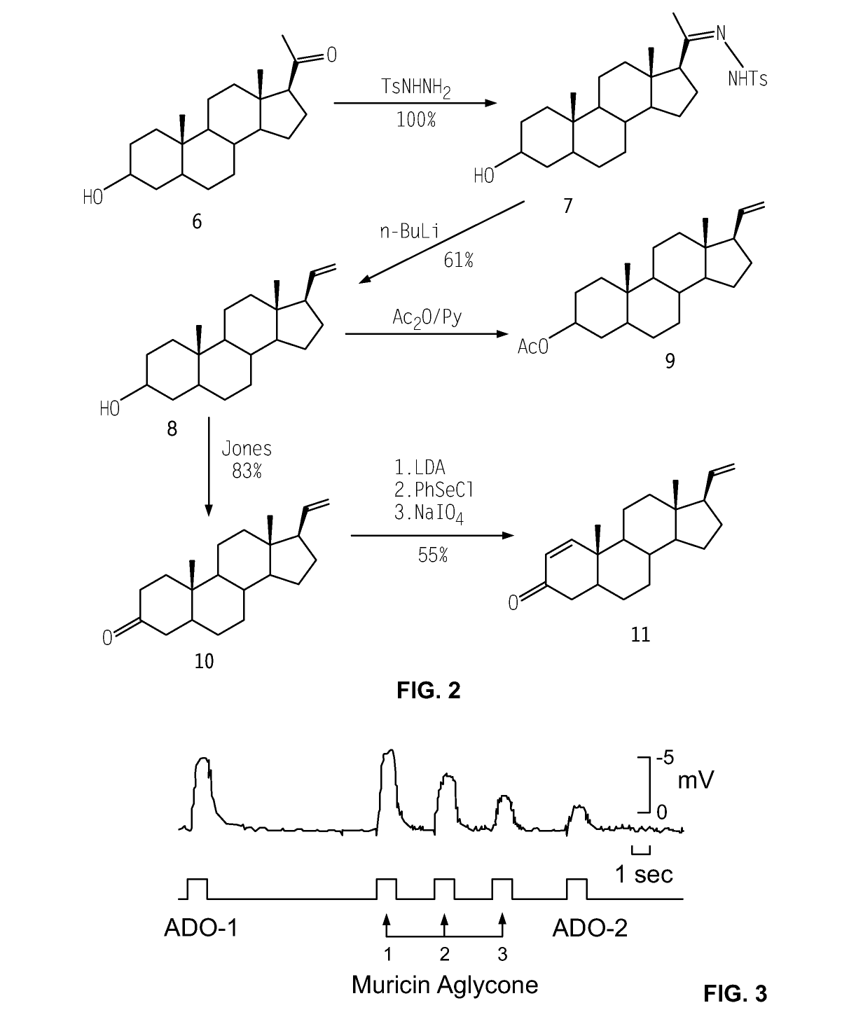

The synthesis of 5α-pregn-20-en-3α-yl acetate (9, 3α5α) is shown in FIG. 2. The pregnenol (8, 3α5α) is prepared as in Example 3, starting with the 3α5α isomer of the pregnanolone (6). The pregnenol (8, 3α5α) is acetylated with acetic anhydride and pyridine in the usual manner, as known to those skilled in the art (see, for example U.S. Pat. No. 5,633,392, which is hereby incorporated by reference in its entirety, particularly with respect to the synthetic methods described therein) to give 5α-pregn-20-en-3α-yl acetate (9, 3α5α).

### Example 5

### Comparison of Effect on Depolarization of Nasal Chemosensory Receptors for Androstadienone (ADO) and pregna-5,20-dien-3β-ol

The effect of pregna-5,20-dien-3β-ol in comparison to ADO on nasal chemosensory receptors were determined as described in L. Monti-Bloch et al., (1994) Psychoneuroendocrinology 19(5-7):673-686.

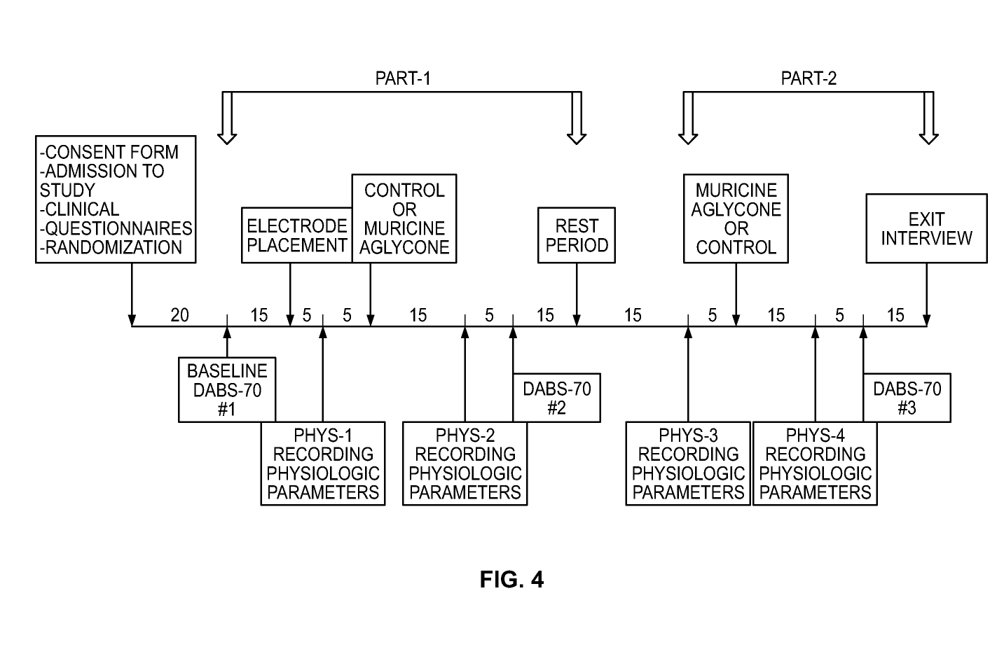

Similar quantities of androstadienone (ADO-1) (27 picograms) and muricin aglycone (pregna-5,20-dien-3β-01) (1) (30 picograms) induced robust depolarization of the electrogram of peripheral nasal chemosensory receptors. Repeated stimulation with muricin aglycone (1, 2, 3) produces fatigue of the receptors as shown by the electrogram induced by subsequently delivered androstadienone (ADO-2), as shown in FIG. 3 (adapted from FIG. 4 of Monti-Bloch et al., (1994) supra). Based on these data, it would be expected that the psychological/behavioral effects of muricin aglycone would be similar to ADO, for example, reduction of negativity and no effect on the levels of social warmth and friendliness, personal well-being, etc. such as described for muricin aglycone in Example 6 below. However, quite surprisingly, as described in the Examples below, muricin aglycone instead showed no effect on negativity and enhanced levels of social warmth and friendliness, personal well-being, etc. as described below.

### Example 6

### Physiological and Psychological Effects of Pregna-5,20-Dien-3β-Ol in Men and Women

The physiologic and psychological effects of pregna-5,20-dien-3β-ol were investigated in men and women. Additionally, the local and general tolerability and safety of pregna-5,20-dien-3β-ol delivered airborne to the nasal passages were also evaluated.

### Methods

We assessed the resulting physiological responses and behavioral changes induced by nasal exposure to 12 ng pregna-5,20-dien-3β-ol in 76 volunteers (38 women and 38 men), age 18 to 46 (Table 1) using a placebo-controlled, single blind and crossover study design. The study design is schematically summarized in FIG. 4. The items of the modified DABS-70 were rearranged during consecutive administrations, in order to minimize the effect of learning.

TABLE 1
Gender, age and number of volunteers participating in the study
Gender

| Men | Women |
|---|---|
| N = 38 | N = 38 |
| Mean Age = 33.9, SDM = 9.4 | Mean Age = 30.9, SDM = 8.9 |
| Median Age = 33.5 | Median Age = 30.0 |

Healthy volunteers, recruited through advertisement in bulletin boards at the University of Utah campus, participated upon signed consent in a single study session lasting 2 hours and 10 minutes. Exclusion criteria for participation were pregnancy, use of psychotropic medication, substance abuse, alcohol abuse, steroidal contraceptives and any neuropsychiatric condition ascertained by interview. At the beginning of the session each volunteer answered a questionnaire about his or her health. Women participants were also requested to complete a brief menstrual calendar.

### Psychometrics

As shown in FIG. 4, at the beginning of the study session (baseline), 20 minutes after the first randomized exposure to pregna-5,20-dien-3β-ol or control, and again 20 minutes after the second randomized exposure (minute 100 of the study), each subject filled out a 67-item modified version, described below, of the psychometric instrument Derogatis Inventory (DABS-70) (Derogatis and Melisaratos, 1979), alone in a quiet room (see also Grosser et al, 2000). Forty of the items on the standard DABS-70 questionnaire are descriptions of “ways people feel”, and subjects indicate whether they had experienced each emotion “never”, “rarely”, “sometimes”, “frequently”, or “always” in the last two weeks. The remaining 30 items on the standard DABS-70 questionnaire are personality characteristics for which subjects describe themselves as “not at all,” “a little bit,” “moderately” “quite a bit” or “extremely,” depending on the degree to which they feel each trait is typical of them. In the modified DABS-70 questionnaire, sixty-seven items were selected and assessed and during the testing the questions were randomized to minimize or prevent “learning” the questionnaire by the volunteers. This psychometric instrument was chosen because several items were of a sexual connotation and could be used to quantify the effects of pregna-5,20-dien-3β-ol on sexuality.

Subjects who participated were asked, as a baseline, to fill out the modified DABS-70 as to their personality characteristics and feelings as they currently perceived them. Each item was quantified ranging from an individual's perception of “not at all” (0) to “extremely” (4). At the beginning of the questionnaire was a statement that they might feel differently after the blind and randomized exposure to the contents of a perfumery grade smelling strip positioned on the upper lip. The baseline electrophysiological measurements were performed after the questionnaire was completed, and again 5 minutes before the second randomized exposure (FIG. 3). Twenty minutes after the first randomized exposure to control or pregna-5,20-dien-3β-ol and again 20 minutes after the second randomized exposure to control or pregna-5,20-dien-3β-ol (in both instances, 5 minutes after the second and fourth set of electrophysiological measurements), the modified DABS-70 was administered again (FIG. 4). This task took approximately 10 minutes to complete, and the order of the questions of each consecutive modified DABS-70 administered was rearranged in order to minimize the effect of learning during prior administration of the test.

### Electrophysiology

Upon completion of the first DABS-70, each subject was invited to rest supine in a comfortable medical bed in a quiet room and several non-invasive electrode leads and transducers were positioned on the surface skin of the arms, face and skull to help measure physiologic parameters (FIG. 4). A conventional grounding electrode and a reference electrode lead were positioned on the surface skin of the mastoid process and the right ear lobe respectively, with the aid of hypoallergenic conductive paste. Other non-invasive standard electrode-leads and transducers were used for recording the following physiologic parameters:
Electrocardiogram (EKG), standard bipolar I.
Electropneumogram (EPG), strain gauge around lower thorax.
Electrodermal activity (EDA), palm surface of 3rd and 4th finger (right hand).
Blinking reflex (BR), upper and lower left eye-lid.
Electromyogram (EMG), bilaterally in chin area.
Body temperature (BT), mini-thermistor probe in external ear canal.
Electroencephalogram (EEG), CzA2 and T4A2 from the 20/20 International EEG electrode placement chart.

During the course of a recording session, physiologic readings were performed in four consecutive instances, before and after randomized administration of active substance or control (FIG. 4): PHYS-1 recording, starting 5 minutes before the first randomized dosing; PHYS-2, starting 15 minutes after administration of the first randomized dosing; PHYS-3, starting 5 minutes before administration of the second randomized dosing; and PHYS-4 starting 15 minutes after administration of the second randomized dosing.

All physiological recordings were pre-amplified and digitized (Biopac Systems), and continuously computer monitored and stored (Macintosh G3), for further off-line processing and analysis (for a detailed explanation see Monti-Bloch et al, 1994, 1998).

### Administration of Pregna-5,20-Dien-3β-Ol and Control (Placebo)

Active substance (pregna-5,20-dien-3β-ol) and control were freshly prepared on 0.7 cm×10 cm perfumery grade smelling strips 24 hours before the experiment. A perfumery grade smelling strip was positioned on the upper lip of the subject for delivery of control or active substance to the nasal passages for a period of 20 minutes. Each breath carried vapors of pregna-5,20-dien-3β-ol or control (air) from the paper strip to the nasal passages.

Perfumery grade smelling strips containing either pregna-5,20-dien-3β-ol or control were prepared as follows: 100 μl of stock solution containing 100 mg pregna-5,20-dien-3β-ol/10 mL ethanol (95%) was added to a clean paper strip. Ethanol was allowed to evaporate overnight in a clean fume hood, and 1 mg of dry crystals of pregna-5,20-dien-3β-ol remained embedded in the dried perfumery grade smelling strip. Control was a perfumery grade smelling strip prepared following the same procedure but the solution was devoid of active substance.

The following assumptions were used to calculate the quantity of pregna-5,20-dien-3β-ol delivered to the nasal passages during a study session: a) the quantity of pregna-5,20-dien-3β-ol arriving to the nasal passages with each breath is ≈40 pg (determined as described below); b) the average respiratory rate at rest in men and women is ≈15 breaths per minute; and c) the subject was exposed to pregna-5,20-dien-3β-ol for 20 min during the entire study session. The total calculated quantity of pregna-5,20-dien-3β-ol delivered to the nasal passages over 20 min (exposure during the study session) was calculated as follows: 40 pg/breath×15 breaths/min×20 min=12000 pg=12 ng.

### Calculation of the Quantity of Active Substance Arriving to the Nasal Passages During Each Breath

The very small amount of an active substance delivered in vapor form to the nasal passages was calculated using the data described in the following experiment:

10 mg of active substance in crystal form (powder) was embedded in a sterile cotton pellet. The cotton pellet was positioned in a sterile glass chamber connected to a source of pulsatile clean (sterile) air. The pulsatile air flow was 1 ml per second. The output of the glass chamber (air saturated with active substance, or vapor pulses) was collected (bubbled) during 48 hours in a test tube containing ethanol. At the end of the 48 hour period ethanol was allowed to evaporate, and the quantity of active substance collected during the 48-hour period was measured. The procedure was performed at room temperature.

### Calculations:

### Part A:

Quantity of substance collected during 48 hours=5.4 microgram
Quantity of substance delivered during 1 hour=0.1125 microgram
Quantity of substance delivered during 1 minute=0.001875 microgram (˜0.2 nanogram)
Quantity of substance delivered per vapor pulse (duration=1 second)=0.000031 (˜0.03 nanogram per second, or 30 picogram per second).

### Part B:

The average human respiratory rate at rest is about 15 breaths per minute.
The average breath duration is about 4 seconds.
The average duration of the inspiratory phase of each breath is about ⅓ of 4 seconds, therefore
The average duration of each inspiratory phase is about 1.3 seconds

### Part C:

Since one 1-second vapor pulse delivers 30 picogram of active substance,
In 1.3-second (duration of inspiratory rate) the quantity of active substance delivered is 39 picogram (˜40 picogram).

The delivery method used in the present study did not comprise pressurized air or any pressurized gas. All the recording procedures were done with the aid of standard, non-invasive electrodes attached to the surface skin using conductive paste, and caused minimal discomfort.

After completion of physiologic recordings and the modified DABS-70 (#3), all surface electrodes were removed and the subject went through an exit interview lasting 5 minutes. At this time the investigator recorded again the arterial blood pressure and also inquired the subject about his or her state of feelings, and also any possible adverse effect appearing during the study session.

### Data Processing and Analysis

Psychometric data analysis: To reduce the number of group comparisons and provide a meaningful framework for pre- to post-effects of the treatment, certain of the 67-items were grouped according to the following conceptual categories:

TABLE 2
Psychometric Categories and Items measured
by Modified DABS-70 Inventory.

| Psychometric Category | Items |
|---|---|
| Social Warmth and | Sympathetic, sentimental, compassionate, loving, |
| Friendliness | gentle, friendly, affectionate, warm |
| Arousal/Excitement | Vigorous, excited, energetic, active, lively, |
| | decisive, independent, adventurous, strong, |
| | dominant, assertive, bold |
| Sensuality | Feline, flirtatious, seductive, passionate |
| Personal Well-Being | Confident, calm, satisfied, relaxed, contented, |
| | pleased |
| Negativity | Sensitive, nervous, regretful, blameworthy, tense, |
| | ashamed, anxious, guilty, remorseful, sad, |
| | irritable, hopeless, resentful, worthless, angry, |
| | miserable, enraged, unhappy, bitter, timid |

Individual mean scores were computed for each of the clusters for the pre- and post-experiment administration of the questionnaire. For example, responses to all of the items conceptualized as social warmth and friendliness for an individual subject prior to the experimental (or control) treatment were added and divided by 8 to produce a mean Social Warmth and Friendliness pre-test score for that individual. These mean scores were analyzed using a 2×2, mixed ANOVA with experimental vs. control as the grouping variable and pre- vs. post-scores as the repeated measure. Based on the a priori hypothesis that mean scores should not differentiate groups prior to experimental treatment but would be expected to differ following administration of the active substance, we examined group by repeated measures interactions for each of the ANOVAs.

In the planning stages of the study, a power analysis was conducted using data from a previous study (Grosser et al, 2000). The sample size of 38 (per gender group), given a large population effect, provided us with adequate power (0.80).

### Physiologic Data Analysis

The mean respiratory rate (RF), heart rate (CF), electrodermal activity frequency (EDA), electromyogram (EMG), eye blinking (BR), body temperature (BT), and alpha, beta and theta frequency bands from the EEG recorded using the 20/20 international electrode placement chart, were computer measured and averaged using AcqKnowledge software (Biopac Systems). EEG spectral analysis was performed using the Fourier transform. The mean value of each physiologic parameter after exposure to control and pregna-5,20-dien-3β-ol were compared within gender group and between gender groups (Nₘₑₙ=38; N_{women}=38) using ANOVA Post Hoc Tests (Statview, SAS).

The following five clusters were extracted from the modified DABS-70: Social Warmth and Friendliness, Arousal and Excitement, Sensuality, Personal Well-Being, and Negativity. The mean value of each cluster obtained during baseline and after exposure to control and pregna-5,20-dien-3β-ol were calculated and compared within gender group and between gender groups (Nₘₑₙ=38; N_{women}=38) using ANOVA.

### Results

Seventy-six healthy subjects (38 men and 38 women), from a total of seventy-eight subjects recruited, complied with all the admission criteria stated in the research protocol and were admitted to the study. Each volunteer participated in one study session lasting 2 hours and 10 minutes where several physiologic parameters and behavioral changes were recorded before and after intranasal exposure to a small dose (12 ng) of airborne pregna-5,20-dien-3β-ol or control, delivered to the nasal passages from a perfumery grade smelling strip positioned on the upper lip. Pregna-5,20-dien-3β-ol was well tolerated by all volunteers of both gender groups and there were no reports of local or general adverse side effects recorded during the intervention and 24 hours after completion of the study session. All participating subjects completed the entire study.

### Behavioral Effects

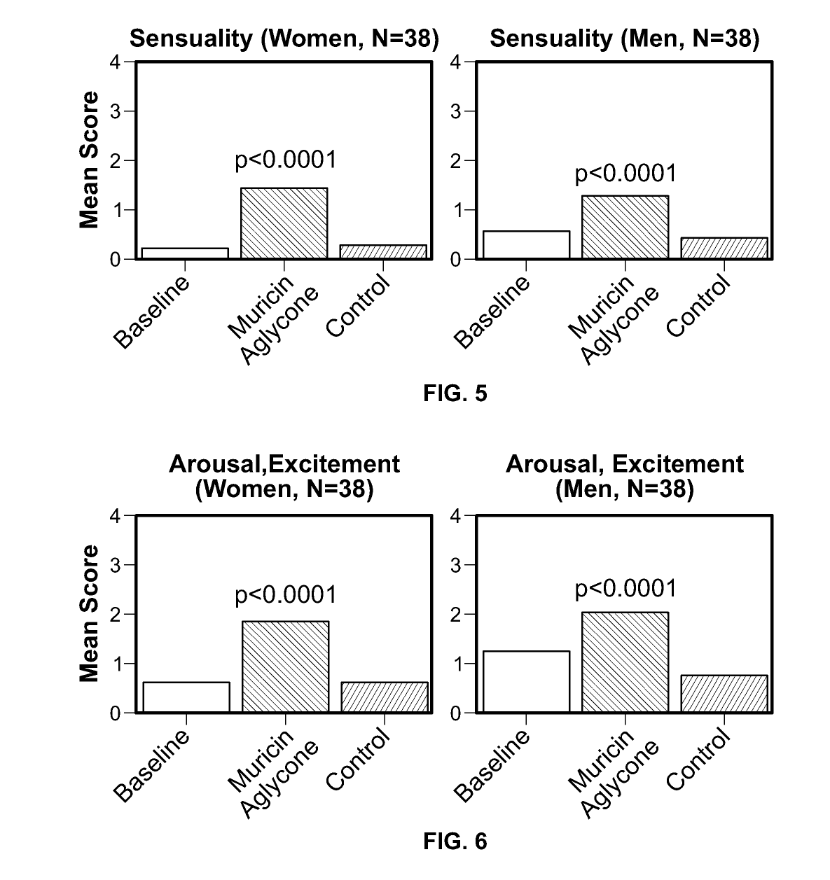

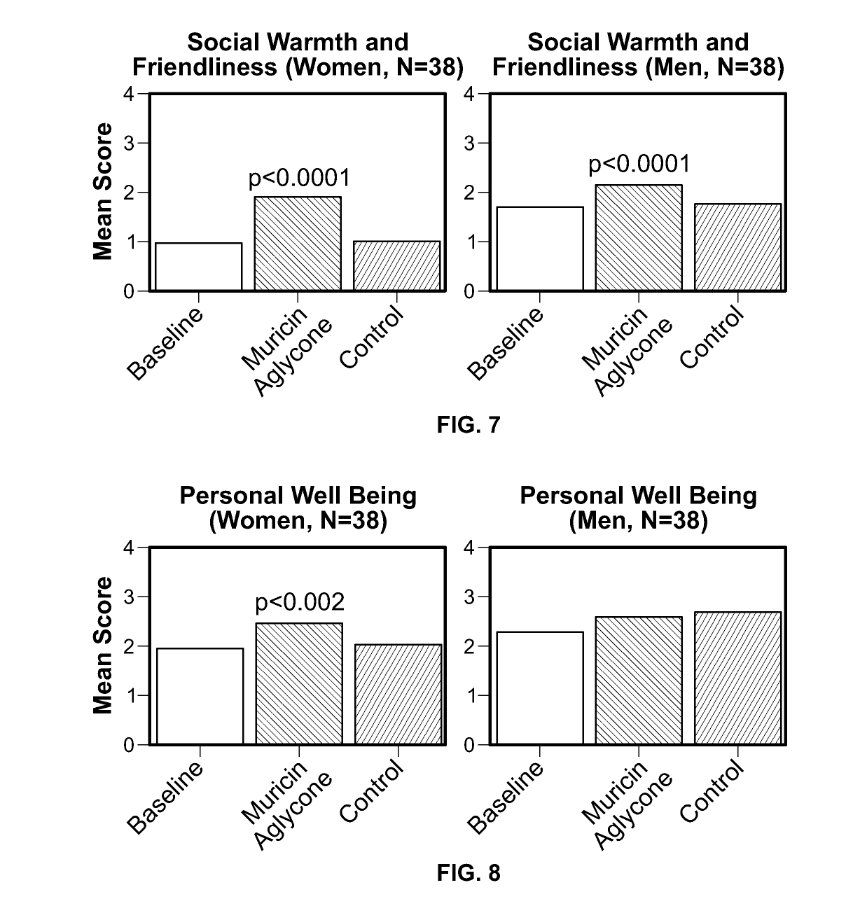

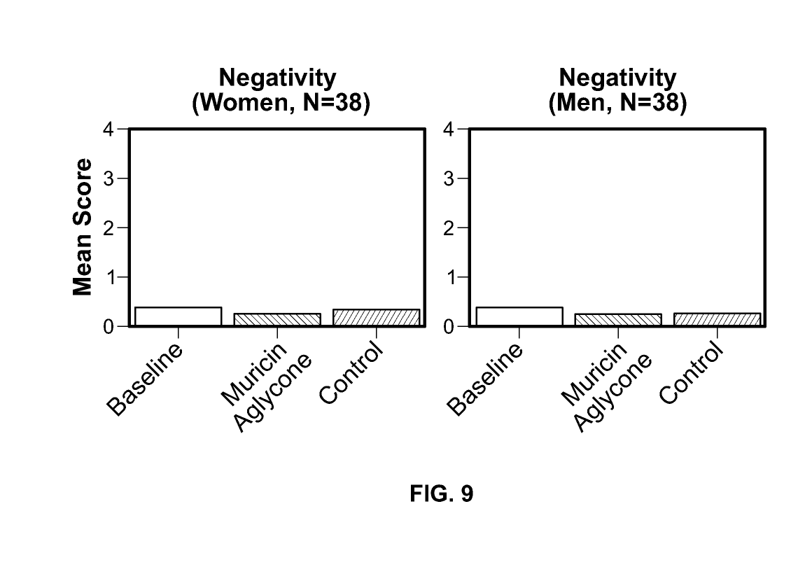

As shown in Table 3 and FIGS. 5-9, there were significant differences in overall responses following exposure to pregna-5,20-dien-3β-ol versus control for the following conceptual clusters: Sensuality (active>control, p<0.0001); Arousal and Excitement (active>control, p<0.0001); Social Warmth and Friendliness (active>control, p<0.00001). These differences were significant regardless of gender

TABLE 3
Behavioral effects of pregna-5,20-dien-3β-ol and control in men (N = 38) and women
(N = 38) using the modified DABS-70 inventory (Independent Group (gender: male,
female) by Repeated Measures (treatment: baseline, active, control) ANOVAs).

| | Means | | | | | |
|---|---|---|---|---|---|---|
| | | | Pregna- | | | |
| | | Baseline | 5,20-dien-3β-ol | Control | | P |
| Sensuality | | | | | Gender | Ns |
| | Women | 0.223 | 1.446 | 0.286 | Treatment | <0.0001 |
| | Men | 0.571 | 1.286 | 0.42 | Interaction | Ns |
| Personal Well-Being | | | | | Gender | <0.0002 |
| | Women | 1.932 | 2.483 | 2.015 | Treatment | <0.002 |
| | Men | 2.304 | 2.607 | 2.66 | Interaction | ns |
| Arousal/Excitement | | | | | Gender | <.002 |
| | Women | 0.613 | 1.857 | 0.602 | Treatment | <.0001 |
| | Men | 1.215 | 2.006 | 0.755 | Interaction | ns |
| Negativity | | | | | Gender | ns |
| | Women | 0.388 | 0.238 | 0.332 | Treatment | ns |
| | Men | 0.392 | 0.246 | 0.269 | Interaction | ns |
| Social Warmth/ | | | | | Gender | <.0001 |
| Friendliness | Women | 0.978 | 1.92 | 0.996 | Treatment | <.00001 |
| | Men | 1.692 | 2.147 | 1.714 | Interaction | ns |

For women only, there was a significant difference in response for the Personal Well-Being cluster (active>control, p<0.002).

Responses to items within the Negativity cluster were not associated with being exposed to active versus control treatment.

In addition to the clusters described above, individual items of the modified DABS-70 inventory showed non-overlapping standard deviations between pregna-5,20-dien-3β-ol treatment and control treatment as follows; in men, pregna-5,20-dien-3β-ol exposure induced a significant increase in “Excited” and “Energetic;” in women, pregna-5,20-dien-3β-ol exposure induced a significant increase in “Excited,” “Loving,” and “Warm,” as shown in the Table 4.

TABLE 4
Individual Modified DABS-70 items wherein there exists a
non-overlap of standard deviations (SD)

| | | | | CON- | | SD |
|---|---|---|---|---|---|---|
| | | ACTIVE | | TROL | | OVER- |
| ADJECTIVE | GENDER | MEAN | SD | MEAN | SD | LAP |
| ENERGETIC | FEMALE | 2.07 | 0.81 | 0.75 | 0.70 | 0.19 |
| ENERGETIC | MALE | 2.32 | 0.77 | 0.86 | 0.65 | −0.04 |
| EXCITED | FEMALE | 1.89 | 0.69 | 0.39 | 0.69 | −0.12 |
| EXCITED | MALE | 2.21 | 0.69 | 0.54 | 0.69 | −0.29 |
| LOVING | FEMALE | 2.11 | 1.03 | 0.05 | 0.92 | −0.11 |
| LOVING | MALE | 2.42 | 0.92 | 1.32 | 1.31 | 1.13 |
| WARM | FEMALE | 2.29 | 0.81 | 0.53 | 0.74 | −0.21 |
| WARM | MALE | 2.25 | 0.80 | 1.71 | 1.08 | 1.34 |

### Physiological Effects

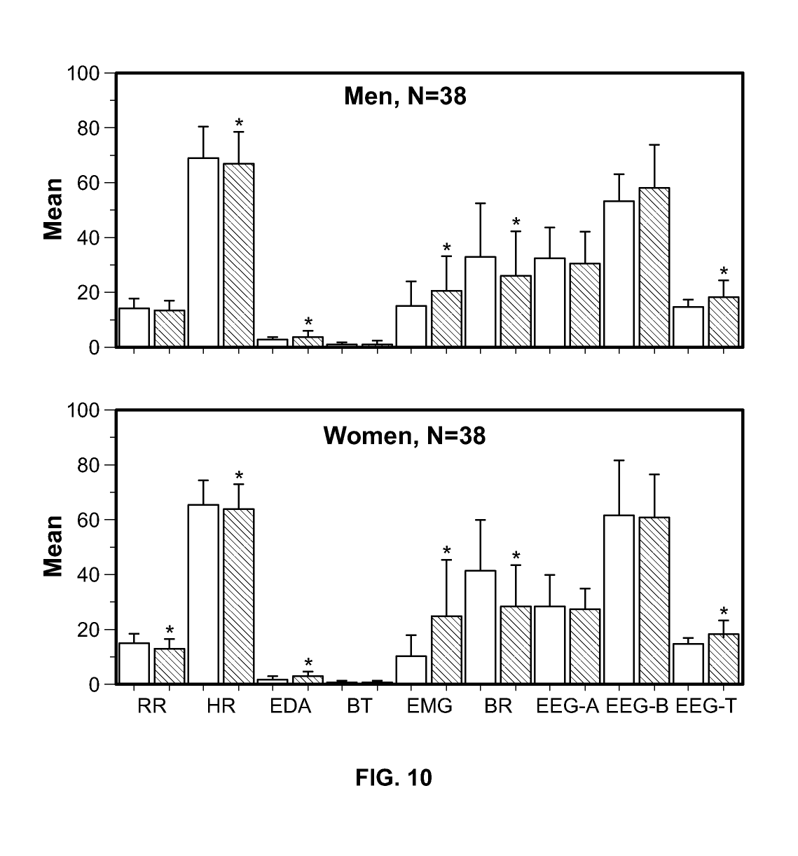

Twenty minutes following the exposure to pregna-5,20-dien-3β-ol, via a paper tape placed on the upper lip of the subject, there were significant changes in physiologic parameters. The mean effects of pregna-5,20-dien-3β-ol and control on physiologic parameters are summarized in FIG. 10 and Tables 5A and 5B. Subjects were exposed to airborne pregna-5,20-dien-3β-ol (12 ng/20 min).

Intranasal exposure to 12 ng pregna-5,20-dien-3β-ol was well tolerated by all volunteers and there were no adverse effects reported. There was no local nasal discomfort and none of the volunteers studied was able to smell pregna-5,20-dien-3β-ol. Clinical examination of the nasal passages at the end of the recording session did not reveal local lesions and/or inflammation of the mucosal lining of the nasal passages. Also, none of the subjects participating in the study reported having local or generalized discomfort during within 24 hours after the recording study, as revealed in a telephone interview the day after they participated in the study.

TABLE 5A
Comparison of effects of pregna-5,20-dien-3β-ol (MA) (12 ng) and control (C) on
physiologic parameters in healthy men volunteers (N-38).

| Physiologic | Mean | | Mean | Mean | | % Change | P | |
|---|---|---|---|---|---|---|---|---|
| Parameter | BL-1 | Mean C | BL-2 | MA | % Change C | MA | C vs. MA | Significance |
| RR | 15.433 | 13.967 | 16.233 | 13.133 | −9.499 | −19.097 | 0.1000 | |
| HR | 69.133 | 68.900 | 68.100 | 66.323 | −0.337 | −2.609 | 0.0002 | S |
| EDA | 2.367 | 2.500 | 2.400 | 3.467 | 5.619 | 44.458 | 0.0130 | S |
| BT | 0.054 | −0.014 | −0.119 | −0.011 | −126.0 | −90.756 | 0.9000 | |
| EMG | 13.987 | 14.387 | 12.197 | 20.067 | 2.860 | 64.524 | 0.0055 | S |
| BR | 37.513 | 32.680 | 33.463 | 25.970 | −12.884 | −22.392 | 0.0290 | S |
| EEG-A | 27.173 | 32.030 | 28.807 | 29.613 | 17.874 | 2.798 | 0.3000 | |
| EEG-B | 69.510 | 52.837 | 67.030 | 57.853 | −23.986 | −13.691 | 0.1500 | |
| EEG-T | 14.260 | 13.927 | 15.460 | 17.630 | −2.335 | 14.036 | 0.0050 | S |

RR = respiration rate,
HR = heart rate,
EDA = electrodermal activity,
BT = body temperature,
EMG = electromyogram,
BR = blinking reflex,
EEG-A = alpha cortical activity,
EEG-B = beta cortical activity,
EEG-T = theta cortical activity.
Columns baseline-1 (BL-1), control (C), baseline-2 (BL-2) and pregna-5,20-dien-3β-ol show the mean value of each physiologic parameter for the group of men (N-38).

TABLE 5B
Comparison of effects of pregna-5,20-dien-3β-ol (MA) (12 ng) and control (C) on
physiologic parameters in healthy women volunteers (N-38).

| Physiologic | Mean | | Mean | Mean | | % Change | P | |
|---|---|---|---|---|---|---|---|---|
| Parameter | BL-1 | Mean C | BL-2 | MA | % Change C | MA | C vs. MA | Significance |
| RR | 16.067 | 15.167 | 15.933 | 13.379 | −5.602 | −16.030 | 0.0002 | S |
| HR | 66.500 | 66.133 | 66.833 | 64.828 | −0.552 | −3.000 | 0.002 | S |
| EDA | 1.933 | 1.933 | 1.967 | 3.379 | 0.000 | 71.784 | 0.0003 | S |
| BT | −0.032 | −0.012 | 0.066 | 0.064 | −62.500 | −3.030 | 0.2 | |
| EMG | 15.467 | 10.200 | 16.250 | 25.276 | −34.053 | 55.545 | <0.0001 | S |
| BR | 40.517 | 41.867 | 41.800 | 28.483 | 3.332 | −31.859 | 0.003 | S |
| EEG-A | 31.233 | 28.633 | 32.567 | 27.724 | −8.325 | −14.871 | 0.9 | |
| EEG-B | 60.533 | 61.800 | 61.700 | 61.069 | 2.093 | −1.023 | 0.8 | |
| EEG-T | 15.067 | 14.700 | 14.433 | 17.897 | −2.436 | 24.001 | 0.001 | S |

RR = respiration rate,
HR = heart rate,
EDA = electrodermal activity,
BT = body temperature,
EMG = electromyogram,
BR = blinking reflex,
EEG-A = alpha cortical activity,
EEG-B = beta cortical activity,
EEG-T = theta cortical activity.
Columns baseline-1 (BL-1), control (C), baseline-2 (BL-2) and pregna-5,20-dien-3β-ol show the mean value of each physiologic parameter for the group of women (N-38).

Pregna-5,20-dien-3β-ol decreased significantly mean respiratory rate in women (% Change_{RR women}=−16.03, P=0.0002) but not in men. Pregna-5,20-dien-3β-ol significantly decreased mean heart rate in men and women (% Change_{CF Men}=−2.6, P=0.002; % Change_{CF Women}=−3.0, P<0.002). Decreased cardiac rate remained within physiologic range and did not have hemodynamic repercussions as revealed by the absence of significant arterial blood pressure difference taken at the beginning of the study session and again during the exit interview.

Exposure to pregna-5,20-dien-3β-ol significantly increased the mean frequency of EDA events and this was significantly different from the effect of control in both gender groups (% Change_{EDA Men}=44.458, P=0.013; % Change_{EDA Women}=71.784, P=0.003). Body temperature did not change significantly from the effect of control after exposure to pregna-5,20-dien-3β-ol in either gender group.

The electromyogram (EMG) recorded from the peri-oral muscles (M. risorius), with the aid of surface skin recording electrodes, increased in both gender groups after pregna-5,20-dien-3β-ol as compared with the effect of control (% Change_{EMG Men}=64.524, P=0.0055; % Change_{EMG women}=55.545, P=0.0001).

Blinking reflex (BR) also decreased in both gender groups after exposure to pregna-5,20-dien-3β-ol (% Change_{BR men}=−22.392, P=0.029; % Change_{BR women}=−31.859, P=0.003).

As shown in FIG. 10 for both gender groups, pregna-5,20-dien-3β-ol induced significant mean increase in the EEG theta frequency band as compared with the effect of control (% Change_{EEGØ-Men}=14.036, P=0.005; % Change_{EEGØ-Women}=24.001, P=0.001). Pregna-5,20-dien-3β-ol did not have a significant effect on the alpha and beta frequency bands of the EEG.

### CONCLUSION

This study shows that stimulation of nasal chemosensory receptors of human volunteers of both genders with a minuscule (nanogram) effective quantity of pregna-5,20-dien-3β-ol can affect behavior and physiologic parameters inducing changes suggestive of sexual arousal. All volunteers participating in the study did not perceive the active substance as an odor. The mechanism by which pregna-5,20-dien-3β-ol produces its behavioral effects is unknown. However, the physiologic decrease of heart rate and eye blinking has been found in other studies to be associated with pleasant psychological states (Tecee, 1992). Also, increased electrodermal activity (EDA), peri-oral muscle activity (EMG) and the Theta-EEG frequency band of the EEG have been described during erotic excitement and sexual excitation (Cohen et al, 1976).

Therefore, pregna-5,20-dien-3β-ol could be considered as a natural substance active in both genders through chemosensory receptors in the nasal passages that can induce a sexual arousal effect without producing olfactory awareness.

### Example 7

### Electrophysiological Effects of Pregna-1,4,20-Trien-3-One in Men and Women

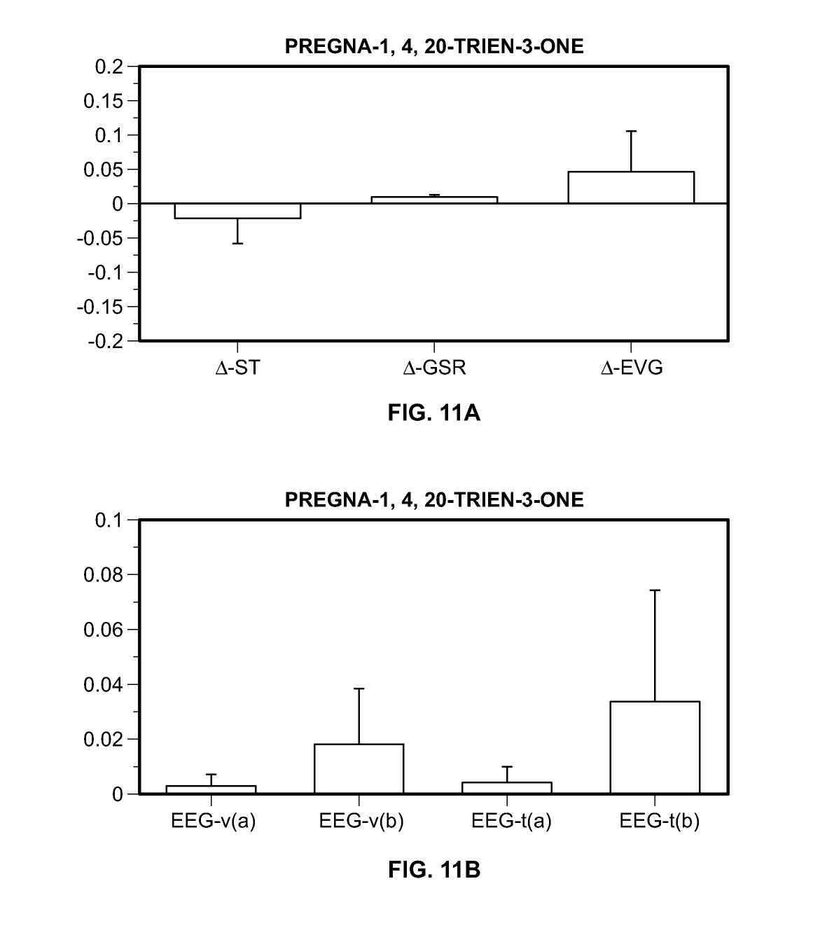

Electrophysiological recordings were conducted by exposure of subjects to pregna-1,4,20-trien-3-one and measuring the response as described in Example 6. The delivery of 29.6 picograms of pregna-1,4,20-trien-3-one was accomplished using the topical probe described in L. Monti-Bloch et al., (1991) J. Steroid Biochemistry & Molec. Biol. 39(4b):573-382 and U.S. Pat. No. 5,303,703 (which are hereby incorporated by reference in their entirety, particularly with respect to the preparation and use of the probes described therein) and the data adapted from U.S. Pat. No. 5,563,131 are shown in FIGS. 11A and 11B.

Based on the data presented in FIG. 11 (A and B) and the similarity of this physiological data to the data obtained in Example 6 (FIG. 10) for pregna-5,20-dien-3β-ol, it is expected that pregna-1,4,20-trien-3-one will have similar effects on the behavior/mood of subjects as pregna-5,20-dien-3β-ol.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entireties for all purposes and to the same extent as if each individual publication, patent, or patent application were specifically and individually indicated to be so incorporated by reference.

### REFERENCES

Andreas si, J. L. (2000). Psychophysiology of human behavior and psychological response. Lawrence Erlbaum Associates, publishers, New Jersey.
Bandurraga et al. (1985). “Isolation of the muricins: evidence of a chemical adaptation against fouling in the marine octocoral Muricea fructicosa (Gorgonacea),” Tetrahedron, 41(6):1057-1065.
Beukes, et al. (1997). “New Polyhydroxylated Pregnadienes from the South African Soft Coral Pieterfaurea unilobata,” J. Nat. Prod., 60:573-577.
Bose et al. (1970). Synthesis, pp. 595-596.
Cohen, H. C. et al. (1976). “Electroencephalographic laterality during human sexual orgasm,” Archives of sexual behavior 5:189.
Dawe et al. (1987). “An improved route to pregna-5,20-dien-3β-ol (muricin aglycone): Carbon and proton nuclear magnetic resonance assignments for the aglycone and a number of related pregnene derivatives,” Can. J. Chem. 65:666-669.
Grosser, B. et al. (2000). “Behavioral and electrophysiological effects of androstadienone, a human pheromone,” Psychoneuroendocrinology, 25:289-299.
Halpert et al. (1989). “Specific Inactivation by 17β-Substituted Steroids of Rabbit and Rat Liver Cytochromes P-450 Responsible for Progesterone 21-Hydroxylation,” Mol. Pharmacol. 35:148-156
Hemrick, M. D. (1974). “Physiological and verbal responses to erotic visual stimuli in a female population,” Behavioral engineering 2:9-16.
Higgs and Faulkner (1977). “5α-Pregna-1,20-dien-3-one and related compounds from a soft coral,” Steroids 30:379-388.
Julian, et al. (1948). “Steroids IV. Δ²⁰-Pregnenes from Bisnor-Steroid Acids,” J. Amer. Chem. Soc. 70:887-892.
Kingston, et al. (1979). “Novel C₂₁Δ²⁰ Pregnanes from the Sea Raspberry (Gersemia rubiformis),” J.C.S. Perkin I, Marine Natural Products, pp. 2064-2068.
Kirte, et al. (1990). “A Survey of the High-field ¹H NMR Spectra of the Steroid Hormones, their Hydroxylated Derivatives, and Related Compounds,” J. Chem. Soc., Perkin Trans. 2, (9):1567-1594.
Klekotka, et al. (1995). “Inactivation of Escherichia coli-Expressed Rabbit Cytochrome P-450 2C Enzymes by 1713-Substituted Steroids,” J. Pharmacol. Exp. Ther. 274:1450-1455.
Krubiner, et al. (1969). “The Synthesis of 17-Deoxy-17α and -17β 20-pregnynes and -20-pregnenes,” J. Org. Chem. 34(11):3502-3505.
Lacey, J. I. et al. (1963). “The visceral level: Situational determinants and behavioral correlates of autonomic response patterns” In Expression of emotions in man, New York, International University Press, pp. 161-196.
Leavitt, F. (1968). “EEG activation and reaction time,” Journal of Experimental Psychology 77:194-199.
Li, et al. (1996). “Synthesis and evaluation of pregnane derivatives as inhibitors of human testicular 17α-hydroxylase/C₁₇₋₂₀-lyase,” J. Med. Chem. 39:4335-4339.
Lindsley, D. B. (1960). “Attention, consciousness, sleep and wakefulness,” Handbook of Physiology, Section I, Neurophysiology, Vol III, pp. 1553-1593.
Maia, et al. (1998). Bol. Soc. Chil. Quin. 43(1):39-45.
Monti-Bloch, L. et al. (1994). “The human vomeronasal system,” Psychoneuroendocrinology (5-7):673-686.
Monti-Bloch, L. et al. (1991). “Effect of putative human pheromones on the electrical activity of the human vomeronasal organ and olfactory epithelium,” J. Ster. Biochem. Molec. Biol. 39(4):573-582.
Monti-Bloch, L. et al. (1998). “The Human Vomeronasal System. A Review.” Olfaction and Taste XII, 855: 373389. The Annals of the New York Academy of Sciences, Nov. 30, 1998
Savic, I., et al (2001). “Smelling of odorous sex hormone-like compounds causes sex-differentiated hypothalamic activation in humans,” Neuron (31):661-668.
Schow et al. (1977). “Synthesis of 5α-Pregna-1,20-dien-3-one,” Steroids 30:389-392.
Sobel, N. et al (1999). “Blind smell: brain activation induced by an undetected air-borne chemical,” Brain, 122:209-217
Tecce, J. J. (1992). Psychology, physiology and experimental psychology. New York, McGraw Hill, pp. 275-377.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. A 20-Pregene-containing composition comprising at least one 20-Pregnene compound having the formula: 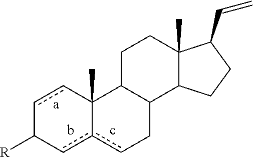
wherein R is selected from the group consisting of: oxo, α-hydroxy, β-hydroxy, α-acetoxy, β-acetoxy, lower α-acyloxy, and lower β-acyloxy;
wherein a, b, and c are individually optional double bonds, with the proviso that b and c are not both double bonds, and with the further proviso that if R is oxo then a is a double bond; and
a composition selected from the group consisting of fragrance compositions comprising an odorant, personal care products, compositions comprising a fibrous material or polymeric component, laundry products, cleaning products, and environmental products.

51. The 20-Pregenene-containing composition according to claim 50, wherein the at least one 20-Pregnene compound occurs naturally in at least one of the following species of coral: Gersemia rubiformis, Carijoa riisei, Pieterfaurea unilobata, or Muricea fruticosa.

52. The 20-Pregenene-containing composition according to claim 50, wherein the at least one compound is selected from the group consisting of: pregna-5,20-dien-3β-ol and pregna-1,4,20-trien-3-one.

53. The 20-Pregenene-containing composition according to claim 50, wherein the at least one compound is pregna-5,20-dien-3β-ol.

54. The 20-Pregenene-containing composition according to claim 50, wherein the at least one compound is pregna-1,4,20-trien-3-one.

55. The 20-Pregene-containing composition according to claim 50, wherein the composition is a fragrance composition and wherein the at least one 20-pregenene compound is present in a non-therapeutic formulation.

56. The 20-Pregene-containing composition according to claim 55, wherein the at least one 20-Pregnene compound occurs naturally in at least one of the following species of coral: Gersemia rubiformis, Carijoa riisei, Pieterfaurea unilobata, or Muricea fruticosa.

57. The 20-Pregene-containing composition according to claim 55, wherein the at least one compound is selected from the group consisting of: pregna-5,20-dien-3β-ol and pregna-1,4,20-trien-3-one.

58. The 20-Pregene-containing composition according to claim 55, wherein the at least one compound is pregna-5,20-dien-3β-ol.

59. The 20-Pregene-containing composition according to claim 55, wherein the at least one compound is pregna-1,4,20-trien-3-one.

60. The 20-Pregene-containing composition according to claim 55, wherein said fragrance composition is formulated for external application to the skin.

61. The 20-Pregene-containing composition according to claim 60, wherein the fragrance composition is a perfume, a mist, a perfumed wax, eau de toilette, or a cologne.

62. The 20-Pregene-containing composition according to claim 55, wherein the compound is present in the composition at a concentration of about 100 ng/ml to about 1 mg/ml.

63. The 20-Pregene-containing composition according to claim 62, wherein the compound is present in the composition at a concentration of about 10 μg/ml.

64. The 20-Pregene-containing composition according to claim 55, wherein the total amount of all odorants comprises about 12% by weight of the composition.

65. The 20-Pregene-containing composition according to claim 50, wherein the composition is a personal care product.

66. The 20-Pregenene-containing composition according to claim 65, wherein the at least one 20-Pregnene compound occurs naturally in at least one of the following species of coral: Gersemia rubiformis, Carijoa riisei, Pieterfaurea unilobata, or Muricea fruticosa.

67. The 20-Pregenene-containing composition according to claim 66, wherein the at least one compound is selected from the group consisting of: pregna-5,20-dien-3β-ol and pregna-1,4,20-trien-3-one.

68. The 20-Pregenene-containing composition according to claim 65, wherein the at least one compound is pregna-5,20-dien-3β-ol.

69. The 20-Pregenene-containing composition according to claim 65, wherein the at least one compound is pregna-1,4,20-trien-3-one.

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)
